SEQUENCE LISTING (2 sequences)

SEQ ID NO: 1 - protein, 340 aa, Homo sapiens
MADSVKTFLQDLARGIKDSIWGICTISKLDARIQQKREEQRRRRASSVLAQRRAQSIERKQESEPRIVSRIFQCCAWNGGVFWFSLLLFYRVFIPVLQSVTARIIGDPSLHGDVWSWLEFFLTSIFSALWVLPLFVLSKVVNAIWFQDIADLAFEVSGRKPHPFPSVSKIIADMLFNLLLQALFLIQGMFVSLFPIHLVGQLVSLLHMSLLYSLYCFEYRWFNKGIEMHQRLSNIERNWPYYFGFGLPLAFLTAMQSSYIISGCLFSILFPLFIISANEAKTPGKAYLFQLRLFSLVVFLSNRLFHKTVYLQSALSSSTSAEKFPSPHPSPAKLKATAGH

SEQ ID NO: 2 - DNA, 2279 bp, Homo sapiens
ccccgcctcgtggtgccggctggttcttcgcgctcgcccgacttcccagcggccccgtgcggcccgggcatgcccagtgcgggcgcagcggccccggccctggaagcgccccggcggagctggcctgcggtgggctaggggcagggccggagccgcggcggcggagctgtggatccttcatgatgagagatttggggacacttctctctcctgtgtgtagttgatagtttggtggtgaagagatggctgacagtgtcaaaacctttctccaggaccttgccagaggaatcaaagactccatctggggtatttgtaccatctcaaagctagatgctcgaatccagcaaaagagagaggagcagcgtcgaagaagggcaagtagtgtcttggcacagagaagagcccagagtatagagcggaagcaagagagtgagccacgtattgttagtagaattttccagtgttgtgcttggaatggtggagtgttctggttcagtctcctcttgttttatcgagtatttattcctgtgcttcagtcggtaacagcccgaattatcggtgacccatcactacatggagatgtttggtcgtggctggaattcttcctcacgtcaattttcagtgctctttgggtgctccccttgtttgtgcttagcaaagtggtgaatgccatttggtttcaggatatagctgacctggcatttgaggtatcagggaggaagcctcacccattccctagtgtcagcaaaataattgctgacatgctcttcaaccttttgctgcaggctcttttcctcattcagggaatgtttgtgagtctctttcccatccatcttgtcggtcagctggttagtctcctgcatatgtcccttctctactcactgtactgctttgaatatcgttggttcaataaaggaattgaaatgcaccagcggttgtctaacatagaaaggaattggccttactactttgggtttggtttgcccttggcttttctcacagcaatgcagtcctcatatattatcagtggctgccttttctctatcctctttcctttattcattatcagcgccaatgaagcaaagacccctggcaaagcatatctcttccagttgcgcctcttctccttggtggtcttcttaagcaacagactcttccacaagacagtctacctgcagtcggccctgagcagctctacttctgcagagaagttcccttcaccgcatccgtcgcctgccaaactgaaggctactgcaggtcactgagttgcctgccatccaaaggggatgggcgggattggaagaagctgtggcagctcttttccctgttcacctcccgcctgccagggaaggcaggacccgctctgccaagggccctctgcgtattcccttctctctgaggaattgaaatttttgtctctggtgcacgtaaggcagaatgttccctgacaccagtgtgtggatttttaacatcaccgtgagtctgaaaggaccacaggtttttctgcagctattttctagcatttgccagtccctgtgcctggactgattggaacactttgtttttctccctgtgccatttacccttccacctttccatcctgccttctaccacccttggatgaatggattttgtaattctagctgttgtattttgtgaatttgttaattttgttgtttttctgtgaaacacatacattggatatgggaggtaaaggagtgtcccagttgctcctggtcactccctttatagccattactgtcttgtttcttgtaactcaggttaggttttggtctctcttgctccactgcaaaaaaaaaaaaaaaaaaaaaaaaaaaaaaaaagcctgaagagatgagataggaggaaagacctcacagccagatctgctgggttttgaggagtgattttctttcttccccttgaaggggaaaaagctattttcattggtacatttaaagtcccccaactatggggaggtaccaattctggacaagtgccactacaacaacactaaacctgaacttttcaactccgttggtggtgggaggcagcgggcagaaatttactgttggccactgccaggtctatttcatatttcaaaggaatattgggtgctgcatataggaactgaaggggtcaatgtattaaacctgtgattggttgttttcctgtcattttgagagactaaatgtggggggcagatgtcaaaatacctgtacaattttaaaatgtcacaattaaacatgagctggtttcccaaaaaaaaaaaaaaaaaa

(19) United States
(12) Patent Application Publication
(10) Pub. No.: US 2018/0028605 A1
(43) Pub. Date: Feb. 1, 2018

(54) PHARMACEUTICAL COMPOSITION FOR THE PREVENTION AND TREATMENT OF CANCER COMPRISING EI24 PROTEIN OR ITS ENCODING GENE

(71) Applicant: Industry-Academic Cooperation Foundation, Yonsei University, Seoul (KR)

(72) Inventors: Han Woong Lee, Seoul (KR); Sushil Devkota, Seoul (KR); Young Hoon Sung, Seoul (KR); Young Jin, Seoul (KR); Wan Je Park, Gyeonggi-do (KR); Yun Mi Kim, Seoul (KR); Jung Min Choi, Incheon (KR); Tae Wook Nam, Seoul (KR); Yu Ra Choi, Gangwon-do (KR)

(21) Appl. No.: 15/659,268

(22) Filed: Jul. 25, 2017

(30) Foreign Application Priority Data
Jul. 26, 2016 (KR) 10-2016-0095043
Jul. 12, 2017 (KR) 10-2017-0088477

(51) Int. Cl. A61K 38/17; G01N 33/50

(52) U.S. Cl. CPC A61K 38/1709; G01N 33/5023; G01N 33/5011; G01N 2333/47

(57) ABSTRACT

Provided herein are a pharmaceutical composition for the prevention and treatment of cancer, including, as an active ingredient, E124 protein or a fragment thereof, or an expression vector including a nucleotide sequence encoding the E124 protein or the fragment thereof, and a method of screening a cancer therapeutic agent candidate.

### CROSS-REFERENCE TO RELATED APPLICATIONS

This application claims priority to and the benefit of Korean Patent Application No. 10-2016-0095043, filed on Jul. 26, 2016 and the benefit of Korean Patent Application No. 10-2017-0088477, filed on Jul. 12, 2017, the disclosures of which are incorporated herein by reference in their entireties.

### BACKGROUND

### 1. Field of the Invention

The present disclosure relates to a pharmaceutical composition for the prevention and treatment of cancer which includes, as an active ingredient, EI24 protein or a fragment thereof; or an expression vector including a nucleotide sequence encoding the EI24 protein or the fragment thereof, and a method of screening a cancer therapeutic agent candidate.

### 2. Discussion of Related Art

p53 known as ‘the guardian of the genome’ is a tumor inhibitory factor. It is very important to maintain the balance of a p53-MDM2-ARF complex in cells because it enables the maintenance of homeostasis and the suppression of a cancer development process. Indeed, the mutation of p53 genes is observed in about 50% of cancer found in humans. This means that the remaining 50% of cancer has genetically undamaged p53 genes, but may develop into cancer while the activity of the p53 protein is inhibited by MDM2-ARF signaling. ARF signaling is known to inhibit p53 from being degraded by MDM2 by bringing MDM2 into the nucleolus.

Thus, once a molecular biological mechanism for recovering p53 functionality is verified, this will be a miracle drug and is expected to be applied as a cancer therapeutic agent. In the related art, as p53-targeting cancer treatment methods, a method of inhibiting the activity of proteasome that degrades p53 and a method of inhibiting the p53-MDM2 interaction using Nutlin-3A, which is a chemical, are used. However, clinical trials having been conducted in previous studies do not show satisfactory results for cancer treatment. Macroautophagy (hereinafter, shortly referred to as autophagy) is an evolutionary process in which cytoplasmic materials are transferred to lysosomes through an autophagosome, which has a structure with double layer membranes and acts as a transport vehicle. Generally, an autophagy mechanism plays a very important role in maintaining the homeostasis of cells, such as degradation of damaged cell organelles. In a nutritionally deficient state, the autophagy mechanism is more activated and catabolism and metabolism, which are self-limited survival mechanisms, are accelerated. The proteasome mechanism that specifically recognizes ubiquitinated proteins and breaks down the proteins and the autophagy mechanism, which is a process whereby cytoplasmic substances are degraded by an autophagosome, are clearly distinct from each other, but the two concepts have not long been distinguished from each other. However, it has been discovered that, in the liquid portion of the cytoplasm, various types of specific autophagy donors such as p62 or Neighbor of BRCA1 gene (NBM1) interact with ubiquitin by UBA and interact with LC3 by an LIR motif, and the autophagic mechanism and the proteasome mechanism have been discovered to be functionally different from each other through this.

To date, an autophagy mechanism that selectively acts on damaged organelles and all proteins or pathogens have been verified and the concept thereof has been partially specified. However, an autophagy mechanism for maintaining physiological functions of cells has not yet been completely specified.

EI24 which is regulated by p53 is a tumor inhibitory factor that inhibits the growth of cancer cells. In addition, the expression of EI24 is known to affect the development of invasive ductal carcinoma and cervical cancer. Recently, it has been verified that EI24 is a gene that plays a vital role in autophagy systems in mice and Caenorhabditis elegans. In addition, the researchers of the present disclosure have discovered in related studies that EI24 inhibits the activity of NF-kB dependently on TRAF2/5, whereby the activity of epithelial to mesenchymal transition (EMT) is inhibited, resulting in carcinogenesis. However, a molecular biological mechanism for how the regulation of EI24 is correlated with autophagy and what important role EI24 plays in functional aspects has not been discovered. However, research into the role of EI24 in the autophagy mechanism or a physiological importance thereof in cancer has not yet been discovered.

Therefore, the inventors of the present disclosure had tried to discover a novel use of

EI24 and, as a result, discovered that EI24 accelerates the degradation of MDM2 by activating an autophagy system in a state in which ARF genes are inactive and, accordingly, the activity of p53 can be recovered, and thus confirmed that EI24 protein or a gene encoding the same can have an inhibitory effect on proliferation of cancer cells through overexpression thereof, thus completing the present disclosure.

### SUMMARY OF THE INVENTION

As described above, the degradation of p53 genes is induced by MDM2 and even when the p53 genes are undamaged, p53 functionality is inhibited, and thus, anticancer agents targeting the recovery thereof have been developed, but satisfactory effects cannot be obtained in the environment where ARF gene functionality is not expressed.

Thus, one or more embodiments of the present disclosure provide a pharmaceutical composition for the prevention and treatment of cancer.

In addition, one or more embodiments of the present disclosure provide a method of screening a candidate for the prevention and treatment of cancer.

According to an aspect of the present disclosure, there is provided a pharmaceutical composition for the prevention and treatment of cancer, including, as an active ingredient, EI24 protein or a fragment thereof; or an expression vector including a nucleotide sequence encoding the EI24 protein or the fragment thereof.

According to one exemplary embodiment, the EI24 protein is a protein having an amino acid sequence of SEQ ID NO: 1, and the gene is a polynucleotide having a base sequence of SEQ ID NO: 2.

According to one exemplary embodiment, in the cancer, ARF gene is non-activated, or expression and activity levels of p53 protein or p53 gene are similar to those of normal cells.

According to one exemplary embodiment, the cancer includes one or more selected from the group consisting of breast cancer, cervical cancer, leukemia, gastric cancer, sarcomas, liver cancer, lung cancer, colorectal cancer, and renal carcinoma.

According to another aspect of the present disclosure, there is provided a method of screening a candidate for the prevention and treatment of cancer, the method including the following processes: treating a patient-derived sample including cells that under-express or are incapable of expressing EI24 protein with test substances; measuring an expression level of the EI24 protein in the treated sample; and selecting a test substance that enables the measured expression level of the EI24 protein to be increased compared to a control not treated therewith.

According to another aspect of the present disclosure, there is provided a method of screening a candidate for the prevention and treatment of cancer, the method including the following processes: treating a sample including EI24 protein with test substances; measuring an activity level of the EI24 protein in the treated sample; and selecting a test substance that enables the measured activity level of the EI24 protein to be increased compared to a control not treated therewith.

According to one exemplary embodiment, in the cancer, ARF gene is non-activated, or expression and activity levels of p53 protein or p53 gene are similar to those of normal cells.

According to one exemplary embodiment, the cancer includes one or more selected from the group consisting of breast cancer, cervical cancer, leukemia, gastric cancer, sarcomas, liver cancer, lung cancer, colorectal cancer, and renal carcinoma.

### BRIEF DESCRIPTION OF THE DRAWINGS

The above and other objects, features and advantages of the present disclosure will become more apparent to those of ordinary skill in the art by describing in detail exemplary embodiments thereof with reference to the accompanying drawings, in which:

FIG. 1 illustrates results showing that EI24 activates an autophagy mechanism;

FIGS. 2A and 2B illustrate results showing that EI24 degrades RINCK1 through the autophagy mechanism;

FIGS. 3A-3C illustrate results showing that EI24 degrades RINCK1 by interacting therewith through an RING domain;

FIGS. 4A-4E illustrate results showing that EI24 can degrade proteins having an RING domain;

FIGS. 5A-5C illustrate prediction results of various types of E3-ligases degraded by EI24;

FIG. 6 illustrates molecular informatics analysis results of targets of EI24 using MPLS-DA;

FIGS. 7A and 7B illustrate results showing changes in protein levels of the predicted various types of E3-ligases according to expression of EI24;

FIG. 8 illustrates results showing that targets of EI24 are mainly positioned in an intracellular area that affects the activity of an autophagy mechanism;

FIGS. 9A-9D illustrate results showing physiological roles of an E3-ligase degraded by an autophagy mechanism and EI24;

FIG. 10 illustrates results showing, as a molecular model, the fact that the autophagy mechanism induced by EI24 degrades MDM2;

FIG. 11 illustrates the fact that most cancer patients with deletion of CDKN2A gene or amplified MDM2 gene have normal EI24 and p53 genes, which demonstrates that EI24 can be a target for cancer treatment;

FIGS. 12A-12C illustrate that EI24 has an LIR-motif and thus can bind to LC3 and activate an autophagy mechanism therethrough;

FIG. 13 illustrates results showing that the activity of EI24 is decreased using EI24 siRNA and thus the autophagy mechanism cannot act.

FIGS. 14A-14C illustrate results showing the fact that EI24 degrades MDM2 through the autophagy mechanism and, accordingly, the expression of p53 is increased;

FIGS. 15A-15C illustrate that EI24 can recognize and bind to an RING domain of MDM2;

FIGS. 16A-16C illustrate results showing that the LIR motif of EI24 binds to the RING domain of MDM2, whereby the autophagy mechanism is activated, resulting in degradation of MDM2;

FIGS. 17A and 17B illustrate results showing that EI24 can maintain the expression of p53 at a constant level;

FIGS. 18A and 18B illustrate that the activity of an E3-ligase by MDM2 is decreased by EI24 and p53 is activated again through this;

FIGS. 19A-19C illustrate analysis results of expression patterns of p53 and ARF in various types of tumor cells;

FIGS. 20A-20D illustrate results that EI24 can inhibit the expression of MDM2 which has the ability to cause cancer;

FIGS. 21A and 21B illustrate results that EI24 maximizes the transcriptional activity ability of p53 under genetic toxic stresses, thereby stopping the G2M phase during cell growth;

FIGS. 22A-22C illustrate the fact that EI24 activates a cancer inhibitory function of p53 in vitro;

FIGS. 23A-23D illustrate the fact that EI24 activates the cancer inhibitory function of p53 in vivo;

FIG. 24A-24C illustrates a tumor growth inhibitory effect according to expression induction of EI24 in MDA-MB-231 cancer cell line-xenografted nude mice;

FIG. 25A-25C illustrates an inhibitory effect of tumor tissue growth according to the expression of EI24 in PyMT cancer model mice;

FIG. 26A-26C illustrates an inhibitory effect of lung metastasis according to the expression of EI24 in PyMT cancer model mice;

FIG. 27 illustrates comparative analysis results of expression patterns of EI24, p53, and MDM2 in various types of cancer patient samples;

FIG. 28 illustrates genes, expression levels of which are varied in cancer tissues that induce the overexpression of EI24 and signaling pathways including the same;

FIGS. 29 and 30 illustrate proteins, expression levels of which are varied according to the overexpression of EI24 in cancer cells; and

FIG. 31 illustrates proteins, activity levels of which are varied according to the overexpression of EI24 in cancer cells.

### DETAILED DESCRIPTION OF EXEMPLARY EMBODIMENTS

Exemplary embodiments of the present disclosure will be described in detail below with reference to the accompanying drawings. While the present disclosure is shown and described in connection with exemplary embodiments thereof, it will be apparent to those skilled in the art that various modifications can be made without departing from the spirit and scope of the invention.

Hereinafter, the present disclosure will be described in detail.

As described above, although p53 gene is undamaged, the degradation of p53 is induced by MDM2 and thus the function thereof is inhibited, and thus anticancer agents targeting the recovery thereof have been developed, but satisfactory effects cannot be obtained in the environment where ARF gene functionality is not expressed.

EI24 according to the present disclosure may degrade MDM2 through an autophagy system and, accordingly, an MDM2-induced decrease in p53 activity may be recovered. This may occur regardless of ARF signaling, and thus enables recovery of p53 functionality through the expression of the EI24 protein even under ARF activity-inhibited or suppressed cancer environments, and may provide cancer treatment effects. Thus, activation of an EI24-mediated mechanism of the present disclosure may play an important role in development of a variety of cancer therapeutic agents.

Therefore, the present disclosure provides a pharmaceutical composition for the prevention and treatment of cancer, including, as an active ingredient, EI24 protein or an expression vector including a gene encoding the same; or an activating agent thereof. The present disclosure also provides a use of EI24 protein or an expression vector including a gene encoding the same; or an activating agent thereof, for the prevention and treatment of cancer. The present invention also provides a method of preventing and treating cancer, including administering, to a patient in need of treatment, EI24 protein or an expression vector including a gene encoding the same; or an activating agent thereof.

The EI24 protein of the present disclosure is composed of an amino acid sequence of SEQ ID NO: 1, and may include the EI24 protein or a functional equivalent to an active fragment thereof.

The term “functional equivalent” as used herein refers to a protein or peptide having substantially the same physiological activity as that of a protein or peptide consisting of amino acid sequences of SEQ ID NOS: 1 to 4, in which the protein or peptide has at least 70%, for example, at least 80%, for example, at least 90%, for example, at least 95%, sequence homology to the amino acid sequences of SEQ ID NOS: 1 to 4, due to addition, substitution or deletion of amino acids of a protein or peptide.

The term “activating agent” as used herein includes a variety of compounds, proteins or peptides, base sequences, and the like which are capable of enhancing the expression of EI24 protein and/or a fragment thereof, or activating an autophagy system by the EI24 protein. The activating agent also includes a variety of metabolites, precursors, or pharmaceutical equivalents of the compounds, proteins or peptides, or base sequences.

The gene of the present disclosure may be a polynucleotide having a base sequence of SEQ ID NO: 2, but the present disclosure is not limited thereto.

The term “cancer” as used herein refers to cancer in which the ARF gene is activated or non-activated, and the cancer may be, for example, cancer in which the ARF gene is non-activated. In a case in which the ARF gene is activated, the ARF protein is capable of binding to MDM2 and thus may inhibit the degradation of p53 by MDM2, but, in the case of cancer in which the ARF gene is non-activated, p53 is degraded by MDM2, and thus p53 cannot function as a cancer inhibitor. Thus, the cancer of the present disclosure may be cancer in which the ARF gene is non-activated.

The cancer of the present disclosure may be cancer in which the expression and activity of p53 protein or gene are similar to those of normal cells, but the present disclosure is not limited thereto. When the expression and/or functionality of p53 are/is lower than those of normal cells, p53 is incapable of effectively functioning as a cancer inhibitor, and thus the cancer of the present disclosure may be cancer in which the expression and/or functionality of p53 are/is similar to those of normal cells.

The cancer of the present disclosure may be one or more selected from the group consisting of breast cancer, cervical cancer, leukemia, gastric cancer, sarcoma, liver cancer, lung cancer, colorectal cancer, and renal carcinoma. For example, the cancer may be one or more selected from the group consisting of colorectal cancer, lung cancer, and liver cancer, but the present disclosure is not limited thereto.

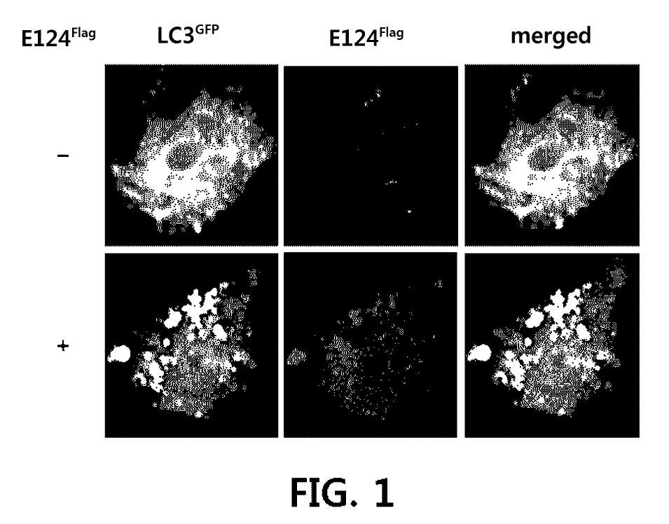

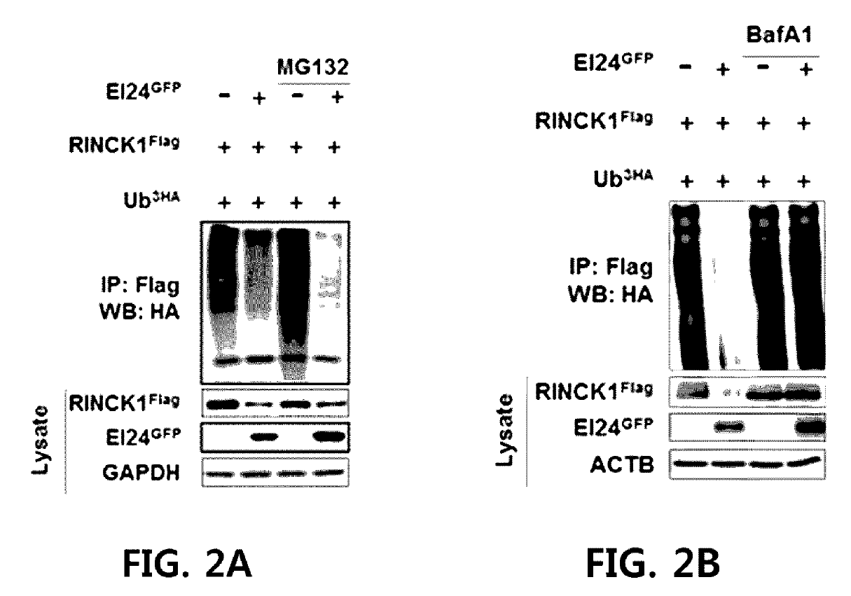

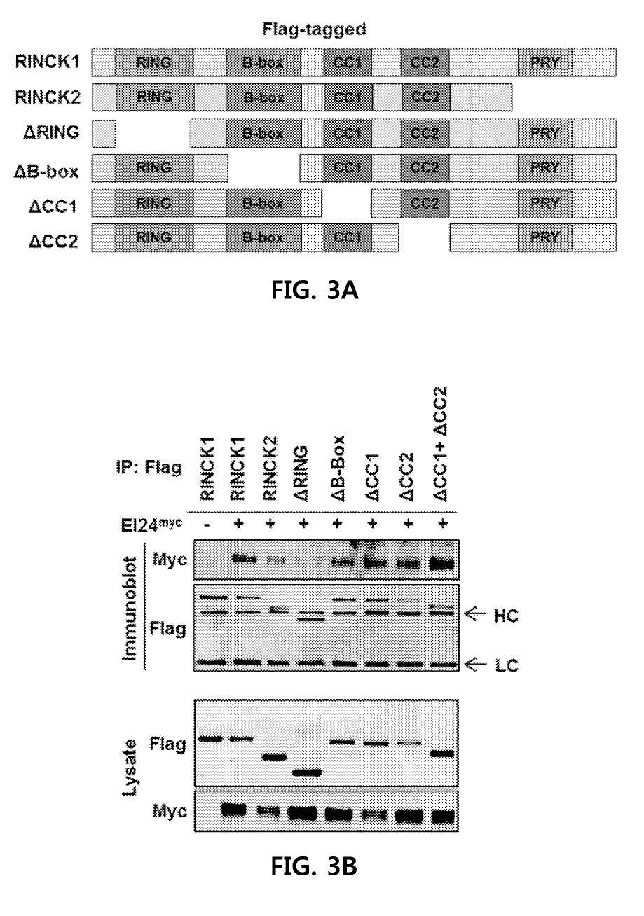

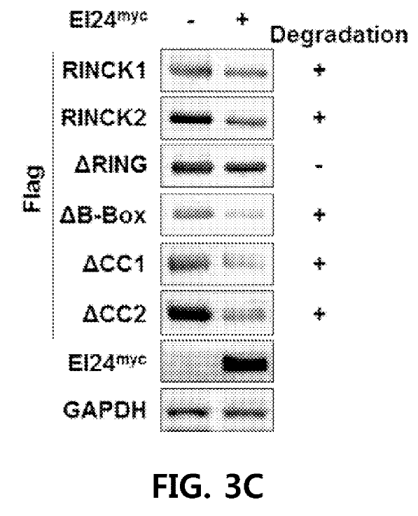

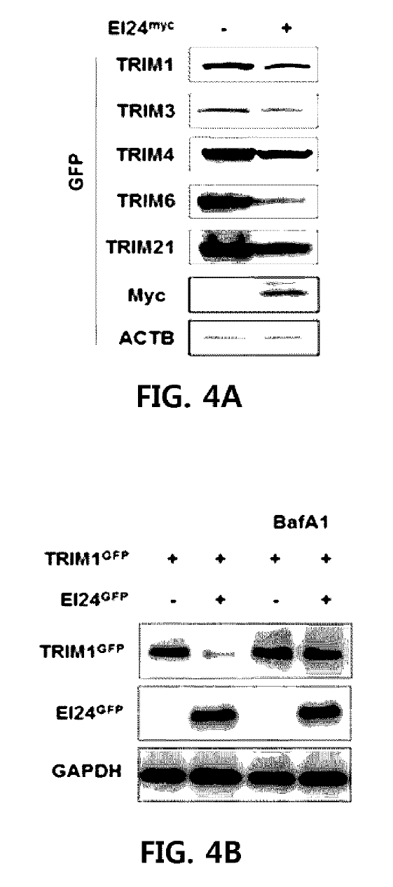

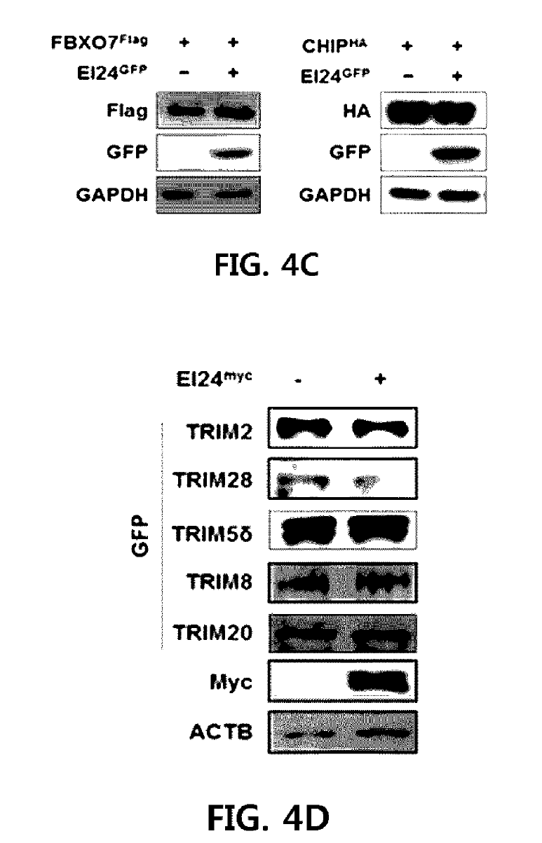

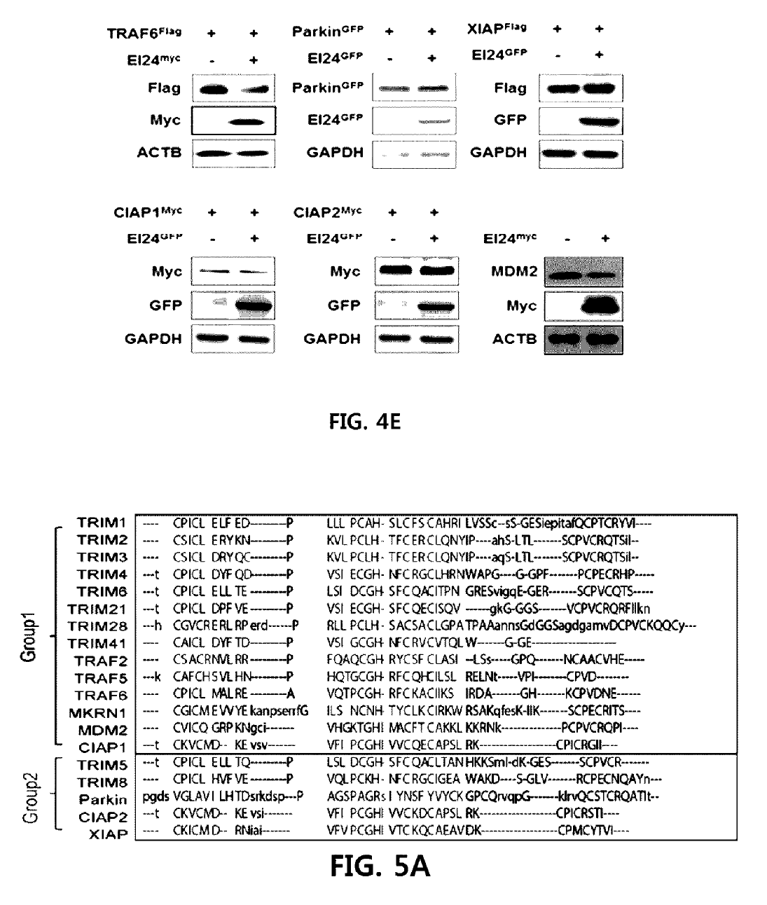

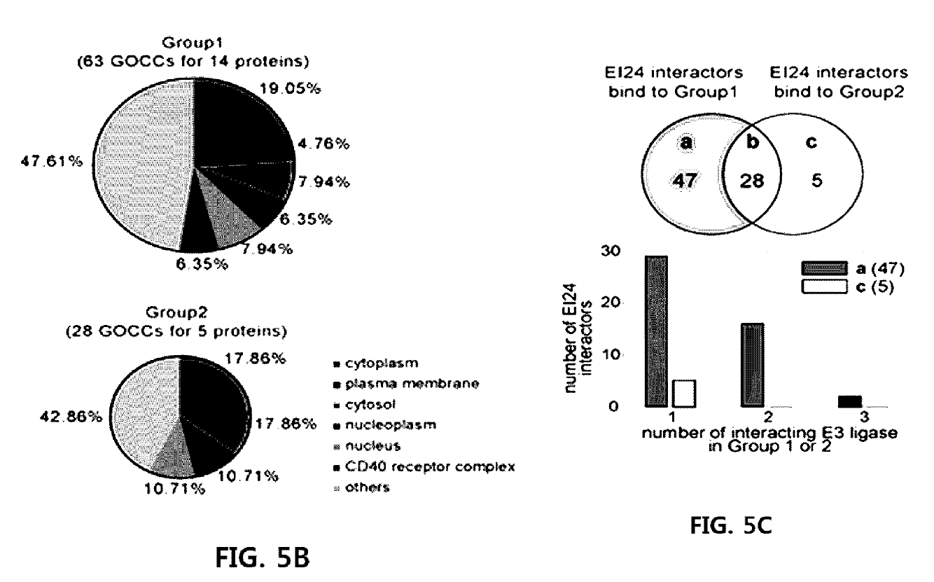

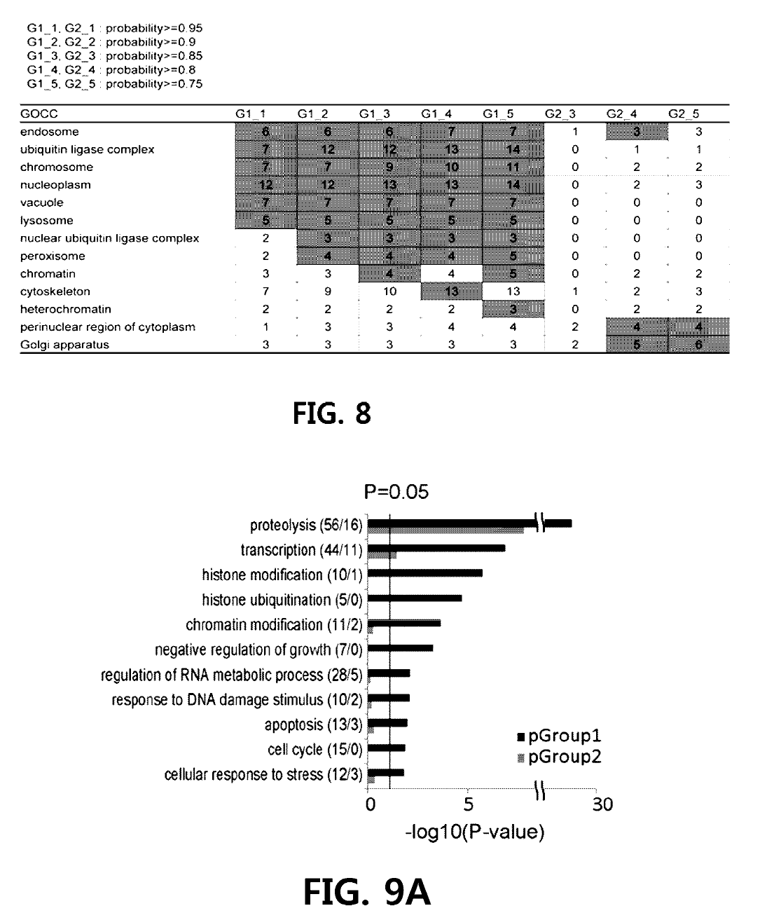

In an exemplary embodiment of the present disclosure, the inventors of the present disclosure confirmed that the EI24 protein could recognize and bind to the RING domain of an E3-ligase by activating an autophagy system (see FIGS. 1, 2A and 2B), thereby degrading the E3-ligase (see FIGS. 3A to 3C and 4A to 4E). Thus, as a result of molecular informatics analysis (see FIGS. 5A to 5C and 8) and functional effectiveness analysis of an E3-ligase which can be autophagically degraded by the EI24 protein, it was confirmed through experiments that EI24 could degrade an E3-ligase having an RING domain through induction of an autophagy system, and a new rule therefor was made through bioinformatics analysis.

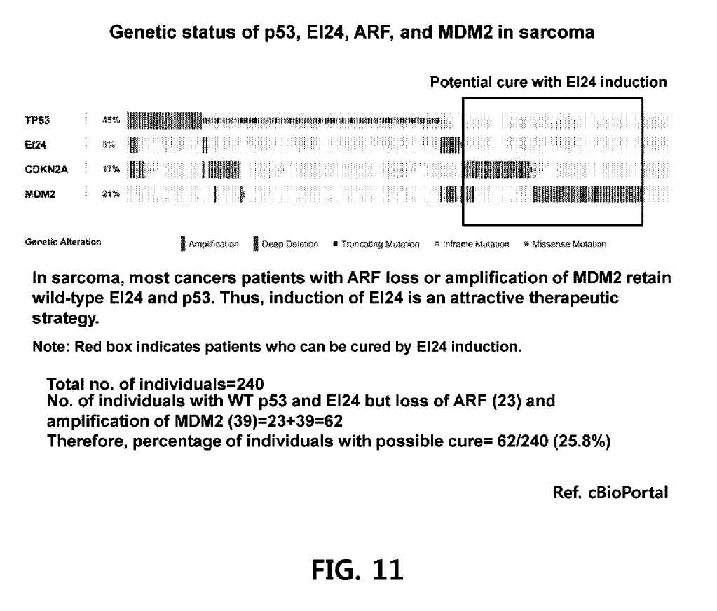

In addition, the inventors of the present disclosure confirmed cancer treatment effects according to the expression of EI24. First, it was confirmed that ARF functionality was lost in 15% of sarcoma patients, and p53 could normally function in 92% of these patients (see FIG. 11).

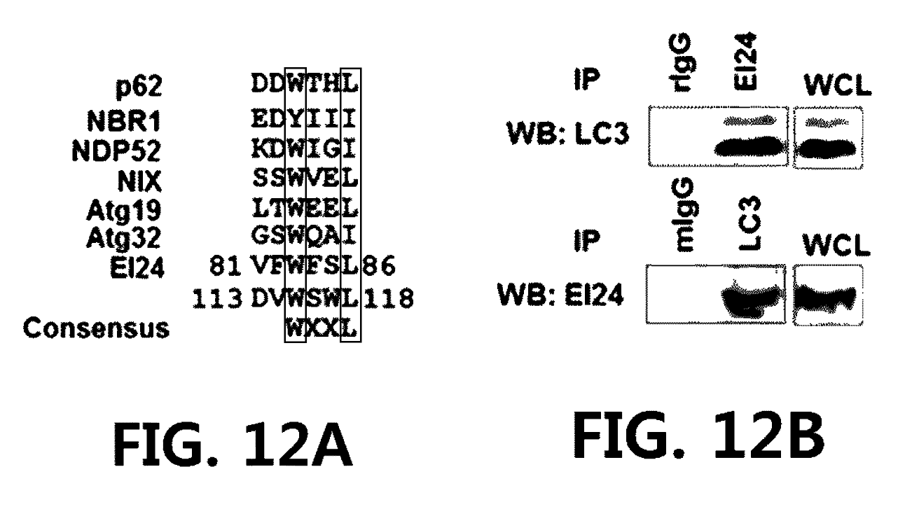

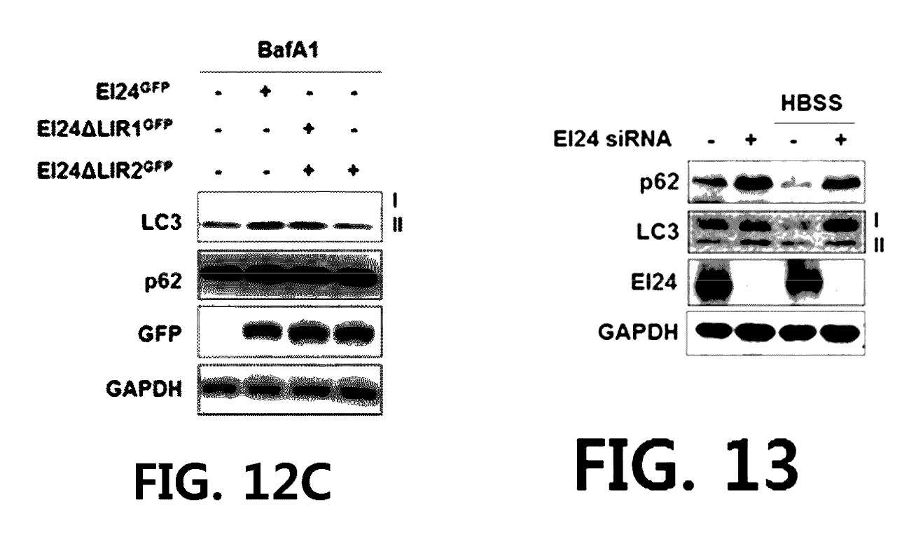

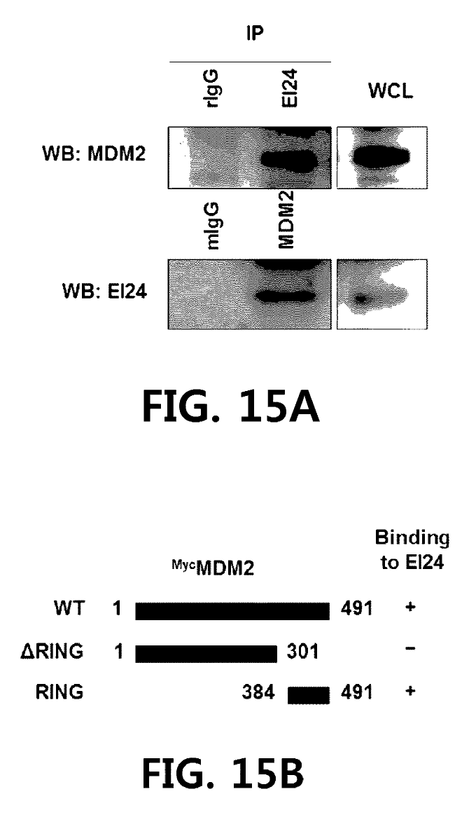

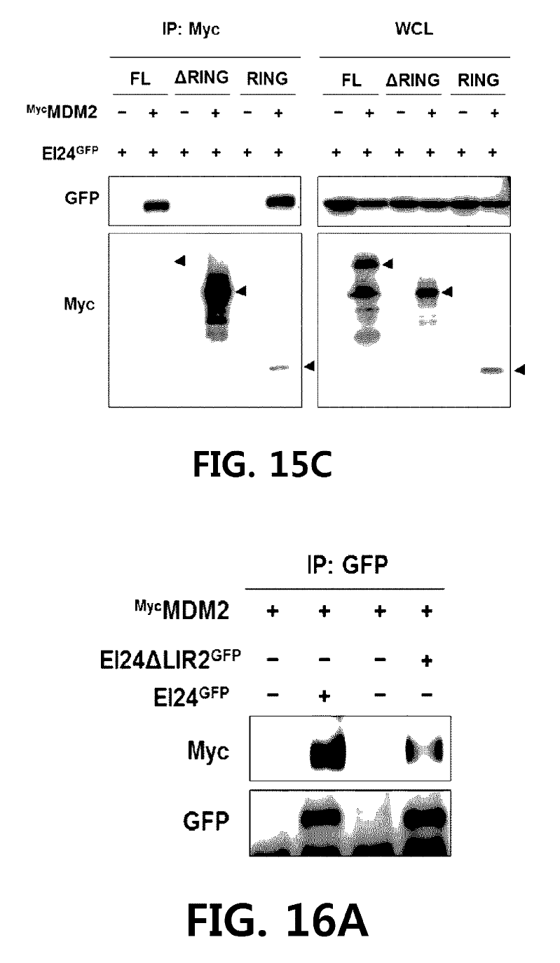

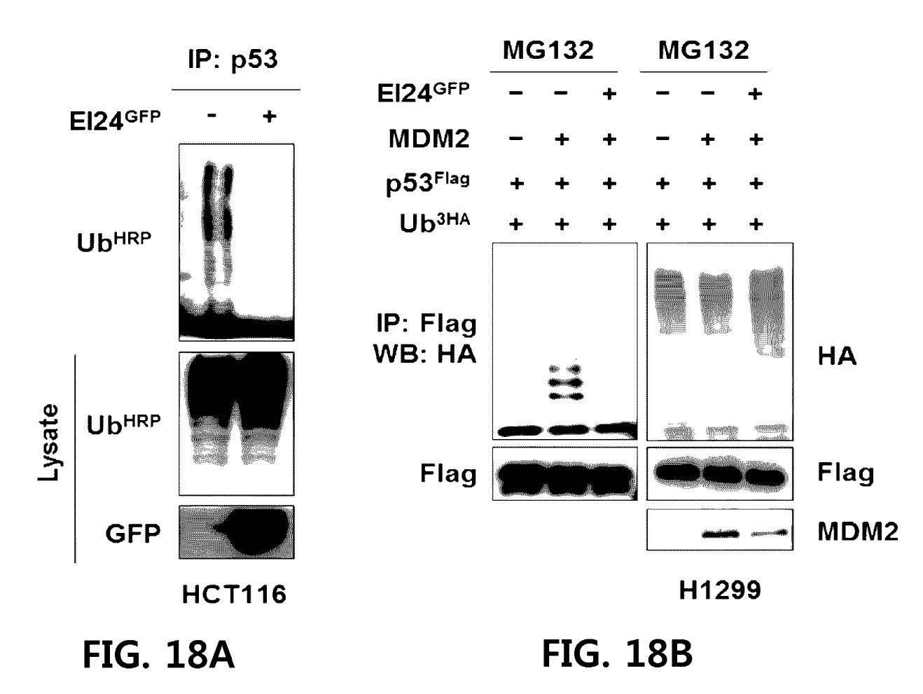

In addition, the inventors of the present disclosure confirmed that autophagic activity was induced according to the presence or absence of EI24 expression (see FIGS. 12A to 12C and 13), and, accordingly, MDM2 was degraded and thus an expression level of the p53 gene was increased (see FIGS. 15A to 15C and 18A and 18B).

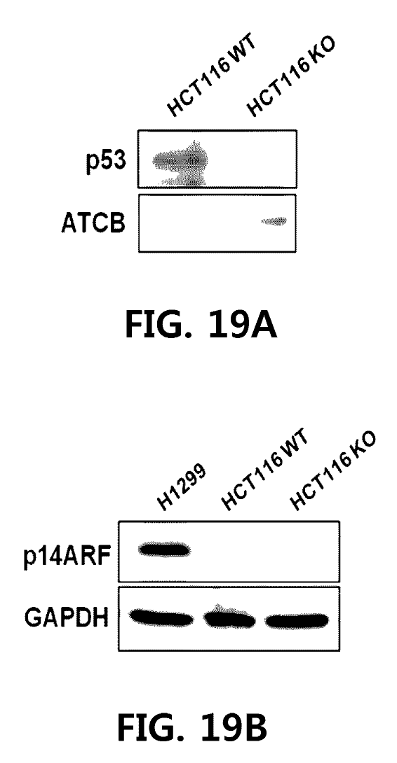

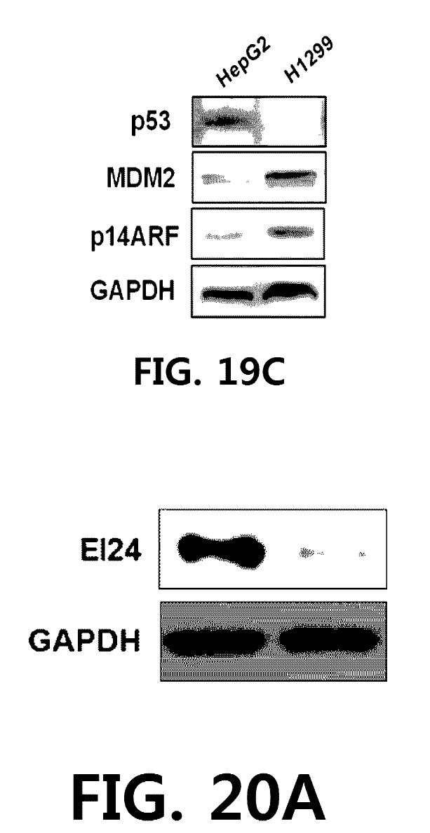

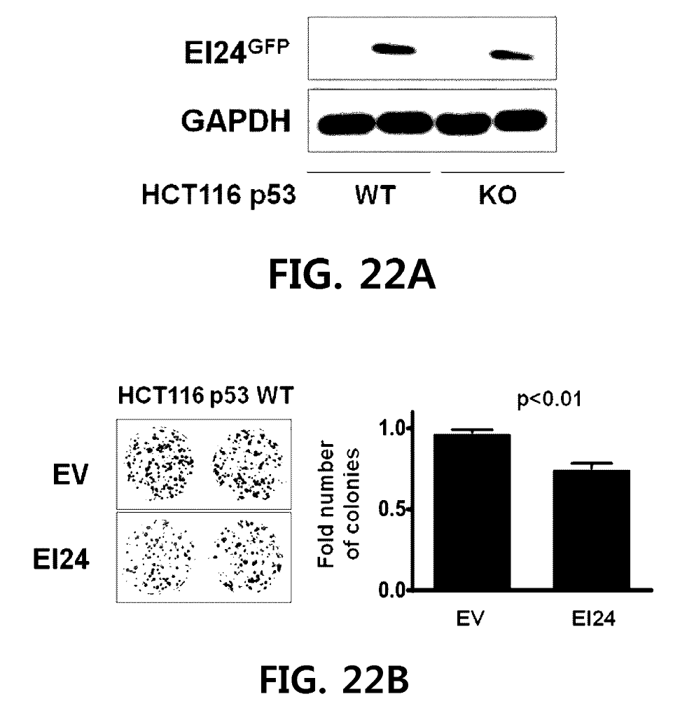

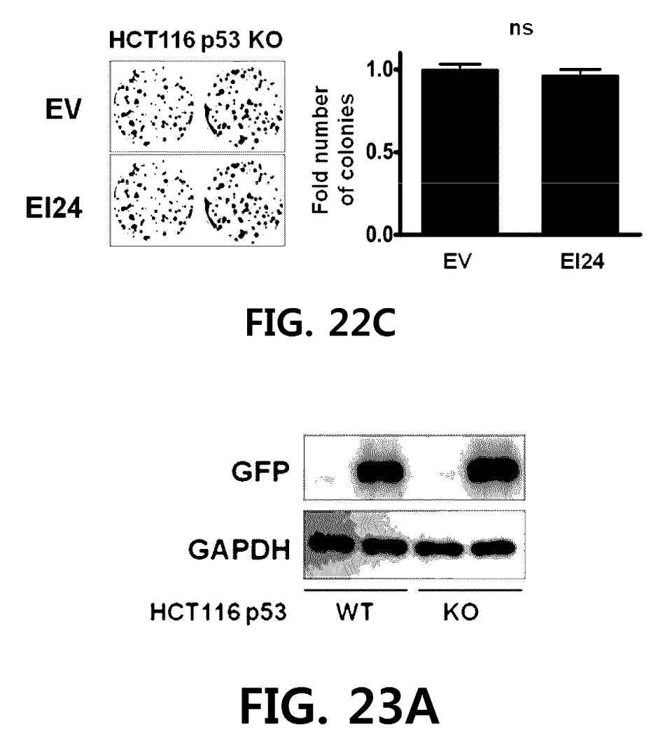

In addition, the inventors of the present disclosure confirmed that, in ARF-expressed or non-expressed cancer cell lines, a proliferation ability of the cancer cell lines was decreased according to the presence or absence of EI24 expression and the viability of tumor cells was decreased according to the recovery of p53 (see FIGS. 19A to 19C and 22A to 22C).

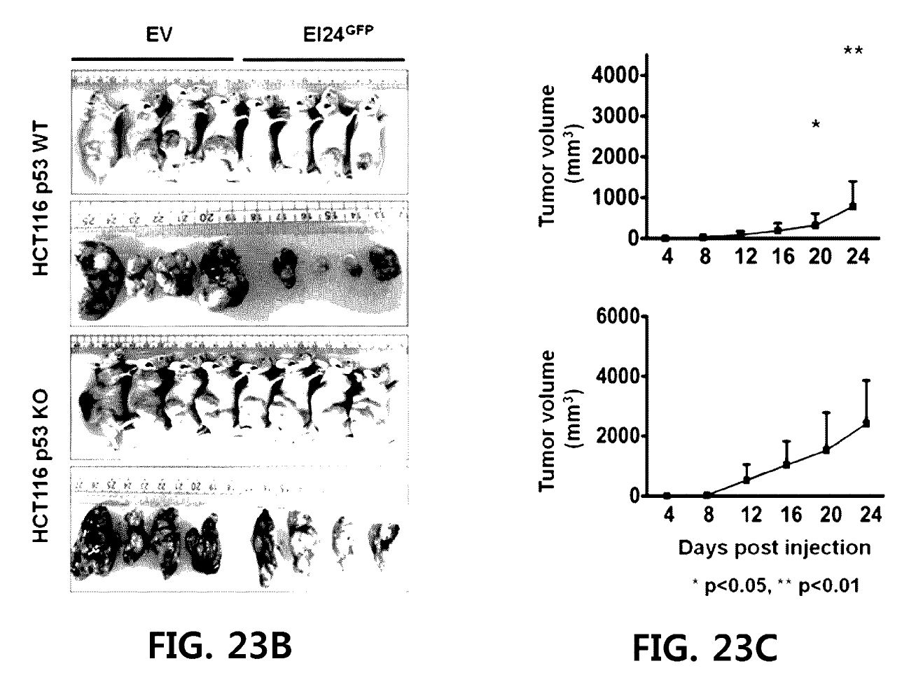

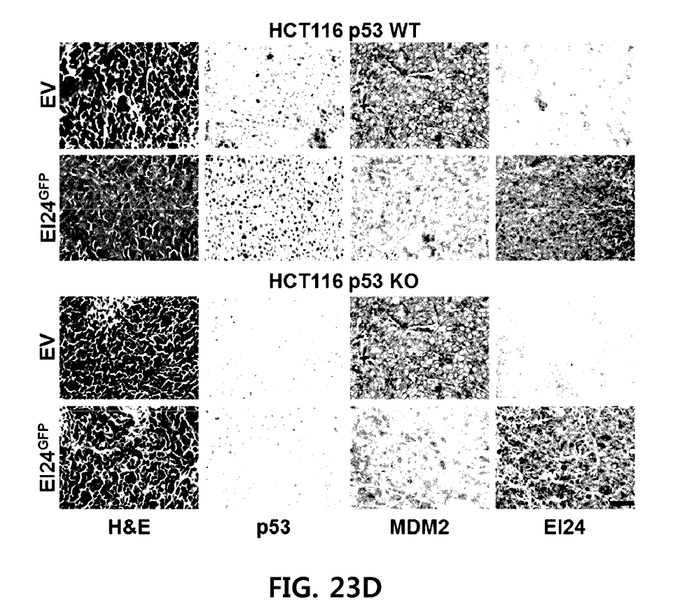

Furthermore, to in vivo confirm the above-described in vitro confirmed results, the inventors of the present disclosure produced tumors in mice and evaluated an effect of EI24 expression on tumorigenesis, from which was confirmed that the growth of cancer was suppressed in the case of EI24-overexpressed cells (see FIGS. 23A to 23D and 27).

Thus, EI24 according to the present disclosure may degrade MDM2 through an autophagy system and, accordingly, an MDM2-induced decrease in p53 activity may be recovered. This may occur regardless of ARF signaling, and thus enables recovery of p53 functionality through the expression of the EI24 protein even under ARF activity-inhibited or suppressed cancer environments, and may provide cancer treatment effects, and, accordingly, EI24 may be usefully used as an active ingredient of a pharmaceutical composition for the prevention and treatment of cancer.

The EI24 protein of the present disclosure may be provided in the form of protein and also in the form of an expression vector capable of expressing a gene encoding EI24 in cells to be used in gene therapies, vaccines, or the like.

The expression vector may be any expression vector known in the art into which a gene encoding the EI24 protein or an active fragment thereof can be inserted to be expressed, for example, an expression vector such as pBK-CMV (Staratagene), pCR3.1 (Invitrogen), or the like.

In addition, the polynucleotide may be administered to a patient to be treated to be expressed in the form in which a recombinant DNA molecule including a base sequence encoding the EI24 protein of the present disclosure, i.e., a polynucleotide is operably linked to a nucleic acid sequence that regulates expression, for example, in the form of an expression vector. Thus, the vector may include a suitable transcription regulation signal including a promoter region capable of expressing an encoding sequence, and the promoter may be operable in a patient to be treated. Thus, the term “promoter” as used herein refers to, in human gene therapy, a promoter which includes sequences needed to direct RNA polymerase to the transcription initiation site, and, if suitable, other operating or regulatory sequences including an enhancer, and the promoter may be a human promoter sequence from human genes or a human promoter sequence from genes generally expressed in humans, e.g., a promoter from a human cytomegalovirus (CMV). In this regard, from among suitable known eukaryotic promoters, the CMV immediate early promoter, the HSV thymidine kinase promoter, the early and late SV40 promoters, the promoters of retroviral LTRs, such as those of the Rous Sarcoma Virus (RSV), and metallothionein promoters, such as the mouse metallothionein-1 promoter, are suitable.

The polynucleotide sequence and transcriptional control sequences may be provided after cloned into a replicable plasmid vector based on commercially available plasmids, such as pBR322, or may be constructed from available plasmids by routine application of well-known published procedures.

The vector may also include a transcriptional control sequence located at the 3′ terminal of the gene sequence, and, when used for human therapy, a polyadenylation sequence recognizable in a patient to be treated, such as the corresponding sequence of a virus such as SV40. The transcriptional control sequences may be any transcriptional control sequences well known in the art.

The expression vector may also include a selectable marker, such as antibiotic resistance, which enables the vector to be propagated.

Expression vectors capable of in situ synthesizing the protein or peptide may be introduced into the wound site directly by physical methods. Examples of these methods include topical application of the “naked” nucleic acid vector in an appropriate vehicle, for example, in a solution in a pharmaceutically acceptable excipient such as phosphate buffered saline (PBS), or administration of the vector by physical methods such as particle bombardment, also known as “gene gun” technology, according to methods known in the art. As described in U.S. Pat. No.: 5,371,015, the “gene gun” technology is a method in which inert particles, such as gold beads coated with a vector are accelerated at a speed sufficient to enable them to penetrate the surface at the wound site, e.g., skin cells, by means of discharge under high pressure from a projecting device. In addition, other physical methods of administering DNA directly to recipients include ultrasound, electrical stimulation, electroporation, microseeding, and the like.

The gene sequence may also be administered to the wound site by means of transformed host cells. Such cells include cells harvested from a patient, and the nucleic acid sequence may be introduced by gene transfer methods known in the art, followed by growth of the transformed cells in culture and grafting to the patient. The expression constructs as described above may be used in the treatment of the present invention using various methods. Thus, the expression constructs may be directly administered to a site of a patient to be treated.

In addition, the pharmaceutical composition of the present invention may include an activation factor for increasing the expression of the EI24 protein or an active fragment thereof.

The activation factor for increasing the expression of the E124 protein or an active fragment thereof refers to a substance that directly or indirectly acts on the E124 gene or a gene encoding an active fragment thereof to thereby enhance, induce, stimulate, and increase the biological activity of the E124 protein or an active fragment thereof. The substance includes a single compound such as an organic or inorganic compound, biopolymers such as peptides, protein, nucleic acids, carbohydrates, and lipids, complexes of multiple compounds, and the like. The activation factor for increasing the expression of the E124 protein or an active fragment thereof may be used in prevention, alleviation, and treatment of diseases that occur due to a decrease in expression, activity, or functionality of the E124 protein. A mechanism whereby the material activates the E124 gene or a gene encoding an active fragment thereof is not particularly limited. For example, the material may increase the expression of the gene, such as transcription, translation, or the like, or may function as a mechanism that converts a non-active type to an active type. For example, the material that activates E124 gene or a gene encoding an active fragment thereof may be a biopolymer such as a peptide, a protein, a nucleic acid, a carbohydrate, and a lipid. As for the E124 protein, nucleic acid and protein sequences of which are already known, a single compound such as an organic or inorganic compound which acts as an inducer or an activator, a biopolymer such as a peptide, a protein, a nucleic acid, a carbohydrate, and a lipid, a complex of multiple compounds, and the like may be prepared or screened by those of ordinary skill in the art using techniques known in the art.

The composition of the present disclosure may be in the form of various oral or parenteral formulations. The composition may be formulated using one or more diluents or excipients, such as a buffer (e.g., saline solution or PBS), an antioxidant, a bacteriostatic agent, a chelating agent (e.g., EDTA or glutathione), a filler, an extender, a binder, an adjuvant (e.g., aluminum hydroxide), a suspension, a thickener, a wetting agent, a disintegrant, or a surfactant.

Examples of solid preparations for oral administration include tablets, pills, powder, granules, capsules, and the like, and these solid preparations are formulated by mixing one or more compounds with one or more excipients, for example, starch (including corn starch, wheat starch, rice starch, potato starch, and the like), calcium carbonate, sucrose, lactose, dextrose, sorbitol, mannitol, xylitol, erythritol maltitol, cellulose, methyl cellulose, sodium carboxymethylcellulose and hydroxypropymethyl-cellulose, gelatin, or the like. For example, tablets or sugar tablets may be obtained by mixing an active ingredient with a solid excipient, pulverizing the mixture, adding a suitable adjuvant thereto, and then formulating the resultant mixture into a granule mixture.

In addition to simple excipients, lubricants such as magnesium stearate, talc, and the like are used. Examples of liquid preparations for oral administration include suspensions, liquids for internal use, emulsions, syrups, and the like, and these liquid preparations may include, in addition to simple commonly used diluents, such as water and liquid paraffin, various types of excipients, for example, a wetting agent, a sweetener, a flavoring agent, a preservative, and the like. In addition, in some cases, a disintegrant such as cross-linked polyvinyl pyrrolidone, agar, alginic acid, sodium alginate, or the like may be added, and an anti-coagulant, a lubricant, a wetting agent, a fragrance, an emulsion, a preservative, and the like may be further added.

Non-limiting examples of preparations for parenteral administration include an aqueous sterile solution, a non-aqueous solvent, a suspension, an emulsion, a freeze-dried preparation, and a suppository. Non-limiting examples of the non-aqueous solvent and the suspension include propylene glycol, polyethylene glycol, a vegetable oil such as olive oil, and an injectable ester such as ethyl oleate. Examples of suppository bases include Witepsol, Macrogol, Tween 61, cacao butter, laurin, glycerogelatin, gelatin, and the like.

The composition of the present disclosure may be administered orally or parenterally, and, when administered parenterally, may be formulated according to a method known in the art in the form of a preparation for external application to the skin; an injection administered intraperitoneally, rectally, intravenously, muscularly, subcutaneously, or intracerebroventricularly, or via cervical intrathecal injection; a percutaneous administration agent; or a nasal inhaler.

The injections must be sterilized and be protected from contamination of microorganisms such as bacteria and fungi. Suitable carriers for injections may be solvents or dispersion media including water, ethanol, polyols (e.g., glycerol, propylene glycol, liquid polyethylene glycol, and the like), mixtures thereof, and/or vegetable oils, but the present disclosure is not limited to the above examples. For example, suitable carriers include, but are not limited to, isotonic solutions such as Hank's solution, Ringer's solution, triethanolamine-containing PBS or sterile water for injection, 10% ethanol, 40% propylene glycol, and 5% dextrose. To protect the injections from microorganism contamination, a variety of antimicrobial agents and antifungal agents such as paraben, chlorobutanol, phenol, sorbic acid, thimerosal, and the like may be further included. In addition, in most cases, the injections may further include an isotonic agent such as sugar or sodium chloride.

Examples of preparations for parenteral administration include ointments, creams, lotions, solution for external use, pastes, liniments, aerosols, and the like. The term “percutaneous administration” as used herein means that an effective amount of the active ingredient of the pharmaceutical composition is delivered into the skin via local administration thereof to the skin.

In the case of preparations for inhalation, the compounds used according to the present disclosure may be conveniently delivered in the form of an aerosol spray from a pressurized pack or a nebulizer by using a suitable propellant, for example, dichlorofluoromethane, trichlorofluoromethane, dichlorotetrafluoroethane, carbon dioxide, or other suitable gases. A dosage unit of the pressurized aerosol may be determined using a valve for transferring the weighed amount. For example, gelatin capsules and cartridges for use in an inhaler or insufflator may be formulated to include a powder mixture of compounds and a suitable powder base such as lactose or starch. Formulations for parenteral administration are described in the document, which is a guidebook generally known in all pharmaceutical chemistry fields (Remington's Pharmaceutical Science, 15th Edition, 1975. Mack Publishing Company, Easton, Pa. 18042, Chapter 87: Blaug, Seymour).

The composition of the present disclosure is administered in a pharmaceutically effective amount. The term “pharmaceutically effective amount” as used herein refers to an amount sufficient to treat diseases at a reasonable benefit/risk ratio applicable to medical treatment, and an effective dosage level may be determined according to factors including type of diseases of patients, the severity of disease, the activity of drugs, sensitivity to drugs, administration time, administration routes, excretion rate, treatment periods, and simultaneously used drugs, and factors well known in other medical fields. The composition of the present disclosure may be administered as an individual therapeutic agent or in combination with other therapeutic agents, may be administered consecutively or simultaneously with existing therapeutic agents, and may be administered in a single dose or multiple doses. That is, the total effective amount of the composition of the present disclosure may be administered to patients in a single dose or may be administered by a fractionated treatment protocol, in which multiple doses are administered over a long period of time. It is important to administer the composition in the minimum amount that enables achievement of the maximum effects without side effects in consideration of all the above-described factors, and this may be easily determined by those of ordinary skill in the art. A dosage of the pharmaceutical composition of the present disclosure varies according to body weight of a patient, age of a patient, gender, body condition, diet, administration time, administration method, excretion rate, and the severity of disease. A daily dosage thereof may be administered parenterally in an amount of about 0.01 mg to about 50 mg, for example, about 0.1 mg to about 30 mg per body weight (1 kg) based on the EI24 protein or an active fragment thereof, and a daily dosage thereof may be administered orally in a single dose or multiple doses in an amount of about 0.01 mg to about 100 mg, for example, about 0.01 mg to about 10 mg per body weight (1 kg), based on the EI24 protein or an active fragment thereof. However, the dosage may be increased or decreased according to administration route, the severity of obesity, gender, body weight, age, and the like, and thus the dosage is not intended to limit the scope of the present disclosure by any method.

The composition of the present disclosure may be used alone or in combination with surgery, radiation therapy, hormone therapy, chemotherapy, and methods using a biological response modifier.

The pharmaceutical composition of the present disclosure may also be provided in the form of a formulation for external use, including the EI24 protein or an active fragment thereof, or a base sequence encoding the same. When used as a preparation for external application to the skin, the pharmaceutical composition for the prevention and treatment of cancer of the present disclosure may further include adjuvants commonly used in dermatology, such as other ingredients commonly used in preparations for external application to the skin, for example, fatty substances, organic solvents, solubilizing agents, thickeners and gelling agents, softeners, antioxidants, suspending agents, stabilizers, foaming agents, fragrances, surfactants, water, ionic or non-ionic emulsifiers, fillers, metal ion blocking agents, chelating agents, preservatives, vitamins, blocking agents, wetting agents, essential oils, dyes, pigments, hydrophilic or lipophilic active agents, lipid vesicles, or the like. In addition, the above-listed ingredients may be introduced in an amount generally used in dermatology field.

When the pharmaceutical composition for the prevention and treatment of cancer of the present disclosure is provided as a preparation for external application to the skin, the preparation may be in the form of a formulation such as ointment, a patch, gel, a cream, an aerosol, or the like, but the present disclosure is not limited thereto.

The present disclosure also provides a method of screening a candidate for the prevention and treatment of cancer, including the following processes:

i) treating a patient-derived sample including cells that under-express or are incapable of expressing EI24 protein with test substances;

ii) measuring an expression level of the EI24 protein in the sample treated in process i); and

iii) selecting a test substance that increases the expression level of the EI24 protein measured in process ii), compared to a control not treated with the test substance.

In process ii) of the present disclosure, the measuring may be performed using any one or more selected from the group consisting of immunoprecipitation, radioimmunoas say (RIA), enzyme linked immunosorbent assay (ELISA), immunohistochemistry, western blotting, and fluorescence activated cell sorting (FACS), but the present disclosure is not limited thereto.

In addition, the method of screening a candidate for the prevention and treatment of cancer of the present disclosure may have a configuration including the following processes:

i) treating an EI24 protein-containing sample with test substances;

ii) measuring an activity level of EI24 protein in the sample treated in process i); and

iii) selecting a test substance that increases the activity level of the EI24 protein measured in process ii), compared to a control not treated with the test substance.

In process ii) of the present disclosure, the measuring may be performed using any one or more selected from the group consisting of sodium dodecyl sulfate (SDS)-polyacrylamide gel electrophoresis (PAGE), immunofluorescence, ELISA, mass analysis, and a protein chip, but the present disclosure is not limited thereto.

In the present disclosure, a reaction between EI24 and the candidate may be identified using one of general methods used to identify the presence or absence of a reaction between a protein and a protein, between a protein and a compound, between DNA and DNA, between DNA and RNA, between DNA and a protein, between DNA and a compound, between RNA and a protein, or between RNA and a compound. Non-limiting examples of the general methods include: an in vitro hybridization test for identifying the presence or absence of binding between the E124 gene and a candidate; Northern blotting after the reaction between a mammalian cell and a test target material; a method of measuring an expression rate of the gene by quantitative PCR, quantitative real-time PCR, or the like; a method of measuring an expression rate of a reporter protein, after linking a reporter gene to the above-described gene to be introduced into a cell and then reacting the introduced gene with a test target material, by quantitative PCR, quantitative real-time PCR, or the like; a method of measuring activity after the reaction between the E124 protein and a candidate; yeast two-hybridization; searching for phage-displayed peptide clones binding to the E124 protein; high throughput screening (HTS) using natural and chemical libraries, or the like; drug hit HTS; cell-based screening; and DNA microarray-using screening.

The term “cancer” as used herein refers to cancer in which ARF gene is activated or non-activated, and the cancer may be, for example, cancer in which ARF gene is non-activated. In a case in which the ARF gene is activated, ARF protein is capable of binding to MDM2 and thus may inhibit the degradation of p53 by MDM2, but, in the case of cancer in which ARF gene is non-activated, p53 is degraded by MDM2, and thus p53 cannot function as a cancer inhibitor. Thus, the cancer of the present disclosure may be cancer in which ARF gene is non-activated.

The cancer of the present disclosure may be cancer in which the expression and activity of p53 protein or gene are similar to those of normal cells, but the present disclosure is not limited thereto. When the expression and/or functionality of p53 are/is lower than those of normal cells, p53 is incapable of effectively functioning as a cancer inhibitor, and thus the cancer of the present disclosure may be cancer in which the expression and/or functionality of p53 are/is similar to those of normal cells.

The cancer of the present disclosure may be one or more selected from the group consisting of breast cancer, cervical cancer, leukemia, gastric cancer, sarcoma, liver cancer, lung cancer, colorectal cancer, and renal carcinoma. For example, the cancer may be one or more selected from the group consisting of colorectal cancer, lung cancer, and liver cancer, but the present disclosure is not limited thereto.

The present disclosure also provides a method of screening a candidate for the prevention and treatment of cancer, including the following processes:

i) treating a patient-derived sample including cells that under-express or are incapable of expressing EI24 protein with test substances;

ii) measuring an expression level of a lower protein of the EI24 protein in the sample treated in process i); and

iii) selecting a test substance by comparing the expression level of the lower protein of the EI24 protein, measured in process ii) above, with that of a control not treated with the test substance.

In addition, the method of screening a candidate for the prevention and treatment of cancer of the present disclosure may have a configuration including the following processes:

i) treating a sample including EI24 protein with test substances;

ii) measuring an activity level of a lower protein of the EI24 protein in the sample treated in process i) above; and

iii) selecting a test substance by comparing the activity level of the lower protein of the EI24 protein measured in process ii) above with that of a control not treated with the test substance.

The lower protein of the EI24 protein refers to a protein, an expression level of which may be varied through regulation of the expression of EI24 gene or EI24 protein or a protein capable of being degraded by the EI24 protein. In particular, the lower protein may include proteins, expression levels of which may be varied by overexpression of EI24 and may be proteins belonging to metabolism, adipogenesis, calcium synthesis, immune responses, and the JAK-STAT signaling pathway.

More particularly, the lower protein of the EI24 protein may be either a) below and b) below or both of them, but the present disclosure is not limited thereto:

a) a protein with a decreasing expression or activity level, which includes any one or more selected from AKT, p-AKT, SRC, pTEM, p-PDK1, P38, pP38, ERK, p-ERK, GSKa/b, p-GSK b, p70 S6K, p-p70 S6K, and HSP60; and

b) a protein with an increasing expression or activity level, which includes any one or more selected from the group consisting of p38a, JNK1/2/3, GSK-3α/β, MSK1/2, CREB, HSP27, AMPKa2, P70 S6K(T389), P70 S6K(T421/S424), and Chk-2.

The term “cancer” as used herein refers to cancer in which ARF gene is activated or non-activated, and the cancer may be, for example, cancer in which ARF gene is non-activated. In a case in which the ARF gene is activated, ARF protein is capable of binding to MDM2 and thus may inhibit the degradation of p53 by MDM2, but, in the case of cancer in which ARF gene is non-activated, p53 is degraded by MDM2, and thus p53 cannot function as a cancer inhibitor. Thus, the cancer of the present disclosure may be cancer in which ARF gene is non-activated.

The cancer of the present disclosure may be cancer in which the expression and activity of p53 protein or gene are similar to those of normal cells, but the present disclosure is not limited thereto. When the expression and/or functionality of p53 are/is lower than those of normal cells, p53 is incapable of effectively functioning as a cancer inhibitor, and thus the cancer of the present disclosure may be cancer in which the expression and/or functionality of p53 are/is similar to those of normal cells.

The cancer of the present disclosure may be one or more selected from the group consisting of breast cancer, cervical cancer, leukemia, gastric cancer, sarcoma, liver cancer, lung cancer, colorectal cancer, and renal carcinoma. For example, the cancer may be one or more selected from the group consisting of colorectal cancer, lung cancer, and liver cancer, but the present disclosure is not limited thereto.

Hereinafter, the present disclosure will be described in further detail with reference to experimental examples and examples. However, these experimental examples and examples are provided only for illustrative purposes and are not intended to limit the scope of the present disclosure.

### EXAMPLES

(Cell Culture)

In the case of mammalian cell lines (embryonic kidney 293T-derived tumor cells and HepG2 hepatic tumor cells), Dulbeco's modified Eagle's medium (DMEM, Gibco, Grand Island, N.Y., USA) supplemented with 10% fetal bovine serum (FBS), 100 units/ml of penicillin, and 100 μg/m of streptomycin (PS) was used. In the case of HCT116 colorectal tumor cells and H1299 lung tumor cells, an RPMI medium supplemented with 10% FBS and PS was used. All the cell lines were incubated in a cell incubator at 5% CO₂ and 37° C. Lipopectamin 2000 was used for transfection, and Lipopectamin RNAiMAX was used for transformation of siRNA. The EI24 knockdown siRNA sequence is 5′-GCAAGAGAGUGAGCCACGUAUUGUUTT-3′.

(Immunohistochemistry)

Tumors produced by xenografting tumor cell lines into immunodeficient mice were separated, fixed with 5 μm paraffin, and sectioned. Expression degrees of p53, MDM2, and EI24 gene in the samples sectioned using xylene were confirmed using a Vecta-stain kit.

(Immunocytochemistry)

Cells were distributed onto gelatin-coated coverslips and fixed with 4% paraformaldehyde. In addition, 0.5% Triton-100 was used to increase permeability. In addition, the cells were blocked using 0.1% Triton X-100-containing 1% normal goat serum, and primary antibodies were allowed to react at room temperature for 1 hour. The primary antibodies were detected using an Alexa-488- or Alexa-568-secondary antibody complex (Invitrogen). Stained cells were identified using a confocal microscope (Zeiss).

(Immunoprecipitation and Western Blotting)

For immunoprecipitation, cells were lysed using a NP-40 buffer (20 mM Tris-HCl, 137 mM NaCl, 1% NP-40, 2 mM EDTA, 10% glycerol, 1 mM PMSF, 2 mM sodium fluoride, 1 mM sodium vanadate, 1 mM β-glycerophosphate, aprotinin/pepstatin/luepeptin 20 μg/ml same amounts) and 1 μg of a primary antibody was added thereto and a reaction was allowed to occur for 3 hours. 30 μl of Protein G agarose beads (Invitrogen) were added thereto and a reaction was allowed to occur for another 3 hours to precipitate a target protein, followed by western blotting.

To collect protein samples, cells were lysed using an RIPA buffer (50 mM Tris-HCl, 150 mM NaCl, 1% NP-40, 0.5% sodium deoxycholate, 0.1% SDS, 2 mM EDTA, and protease inhibitors and phosphatase inhibitors),

(Ubiquitination Assay)

30 hours after transfection, cells were harvested using PBS with 2 mM N-ethylmaleimide (NEM) and were lysed using Tris-buffer saline (TBS) containing 1% SDS and 20 mM NEM. The lysate was boiled and then sonicated and centrifuged, and the supernatant was diluted with an NP-40 buffer containing 2 mM NEM, followed by immunoprecipitation.

(RNA Isolation and Measurement of Expression Amount)

Total RNA was isolated from mouse tissue or a tumor cell line using TRIzol (Invitrogen) and converted to cDNA using the Superscript III First-Strand Synthesis System with Oligo-dT primers (Invitrogen). Expression amounts of genes were measured using IQ SYBR Green SuperMix and quantitatively analyzed using iQ5 optical system software (Bio-Rad).

(Cell Viability Assay)

HepG2 liver tumor cells were transformed with an empty vector (Control), an MDM2 expression vector, and an MDM2 & Myc tagged EI24 expression vector to prepare cells overexpressing MDM2 or EI24 protein, and then 1×10⁵ cells were distributed into cell culture dishes, and the growth rates of the cells were observed on day 0, day 2, day 4, and day 6, respectively. Each cell sample was stained with crystal violet to observe colonies, destained with 10% acetic acid, and isolated, and absorbance thereof was measured at 590 nm.

(Flow Cytometry)

Flow cytometry was performed using FACS Calibur apparatus 342975 (BD Biosciences, San Jose, Calif., USA). Cells were fixed in 70% ethanol and then digested with RNase A (Sigma-Aldrich, R4875) and DNA was stained using propidium iodide (Sigma-Aldrich, P4170). The G1, S, G2, and M phases of the cell cycle were expressed using glyceraldehyde-3-phosphate dehydrogenase (GAPDH).

(Tumor Cell Xenografting Into Immunodeficient Mice)

HCT116 colorectal tumor cells were transfected with an empty vector (Control) and a GFP-tagged EI24 expression vector to prepare cells overexpressing EI24 protein, and then 5×10⁶ cells were mixed with Matrigel in a ratio of 1:1 and the resulting cells were xenografted into immunodeficient mice. The xenografted cells were observed for 20 days and the size of a tumor was measured using a caliper at intervals of two to three days. The mice experiment was carried out after receiving approval from the Institutional Animal Care and Use Committee (IACUC) at Yonsei University and was performed in a feeding room without a specific pathogen.

(Measurement of Half-Life of Cells)

MDM2 and p53 were overexpressed in HCT116 colorectal tumor cells and treated with 10 μg/ml of cycloheximide according to periods of time, and expression degrees thereof were evaluated by western blotting.

(Cancer Genomics and the Cancer Genome Atlas (TCGA) Data Analysis)

To analyze the presence or absence of the expression of EI24, p53, and MDM2 genes and copy number variation patterns in patients, the Oncomine Premium Edition Database (Compendia Biosciences, USA; www.oncomine.org) was used. In addition, cancer genomics algorithms were used for statistical analysis of gene expression. The presence or absence of mutation of the p53 and ARF genes was analyzed using the TCGA database.

### EXAMPLE 1

Confirmation of Activation of Autophagy System of EI24 Protein

<1-1> Confirm Whether Autophagy is Activated in EI24-Expressed Cells

EI24 is known as a vital factor in autophagy systems of mice and Caenorhabditis Elegans. Thus, to specify the role of EI24 in an autophagy mechanism, first, it was checked whether or not autophagy occurred in EI24-expressed cells.

As a result, as illustrated in FIG. 1, when EI24 was overexpressed in a cancer-derived cell line, the number of LC3 dots labeled with GFP increased, from which it was confirmed that the autophagy mechanism was activated (see FIG. 1).

<1-2> Confirmation of E3-Ligase Degradation Mechanism by EI24 Expression

To confirm whether EI24 is capable of degrading a protein through an autophagy system, EI24-overexpressed cells were cultured in a medium treated with MG132, which is a proteasome inhibitor, or BafA1, which is an autophagosome inhibitor, and a change in the expression level of RINCK1, which is an E3-ligase degraded by EI24, was checked.

As a result, as illustrated in FIGS. 2A and 2B, it was confirmed that, when EI24 was overexpressed, the expression of RINCK1, which is an E3-ligase, was decreased. In particular, the expression of RINCK1 was decreased in EI24-mediated protein degradation, regardless of whether to be treated with MG132 (specific, potent, reversible and cell permeable proteasome inhibitor), and there was no difference in an expression amount of RINCK1 between the group treated with BafA1 (obstruction of autophagosome-lysosome fusion by inhibition of dSERCA) and a control. From the results, it was confirmed that autophagy was activated in the EI24-mediated protein degradation process and this affected the proteasome system (see FIGS. 2A and 2B).

<1-3> Confirmation of Effect of EI24 on E3-Ligase With RING Domain by Activating Autophagy System

To confirm a domain of RINCK1, which is an E3-ligase, with which EI24 interacts, as illustrated in FIG. 3A, RINCK1 constructs, from which various domains of RINCK1 were removed, were prepared, and the presence or absence of interaction therebetween was checked by immunoprecipitation.

As a result, as illustrated in FIGS. 3A to 3C, it was confirmed that EI24 interacted with RINCK1, which is an E3-ligase. In addition, the role of domains of RINCK1 was screened and it was confirmed that the RING domain is very important in interaction with EI24.

Based on these results, various types of E3-ligases having the RING domain were screened and it was checked whether these were degraded when EI24 was overexpressed in the same manner. As a result, as illustrated in FIGS. 4A to 4E, it was confirmed that EI24 degraded an E3-ligase by recognizing and binding to the RING domain and this was induced by an autophagy mechanism. However, E3-ligases that were not targeted by EI24 were found in the screening process. Thus, comparative analysis was performed on the above-described phenomena using molecular informatics technology.

### EXAMPLE 2

Molecular Informatics Analysis and Prediction Results of E3-Ligases Degraded by E124

E3-ligases degraded by EI24 were grouped into Group 1, E3-ligases that could not be degraded by EI24 were grouped into Group 2, and molecular informatics analysis was performed to evaluate characteristics of each group.

As a result, as illustrated in FIGS. 5A to 5C, when multiple sequence alignment analysis was performed, Groups 1 and 2 exhibited a high similarity regarding the RING domain, and thus there was no distinct difference in a sequence motif. In addition, it was confirmed that there was no distinct difference between Groups 1 and 2 in terms of the position thereof on cells and analysis of interactomes.

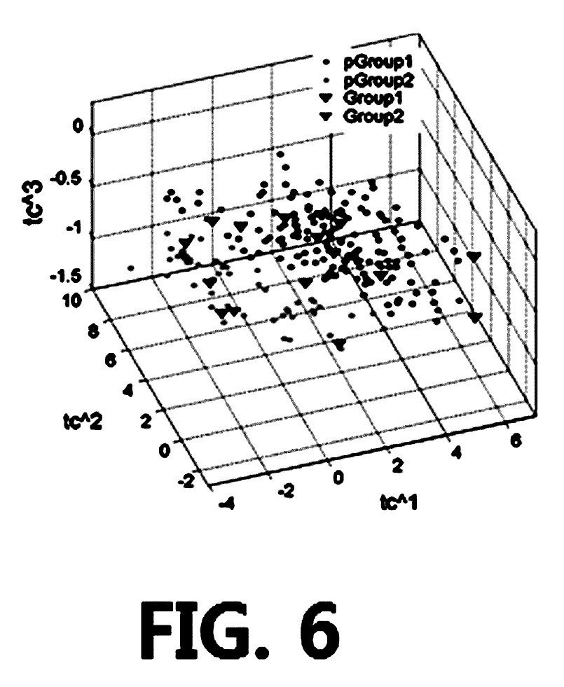

In addition, as a result of analysis of various types of E3-ligases using a metabolomics and partial least squares-discriminant analysis (MPLS-DA) system, as illustrated in FIG. 6, 161 E3-ligases were predicted as Group 1 and 64 E3-ligases were predicted as Group 2.

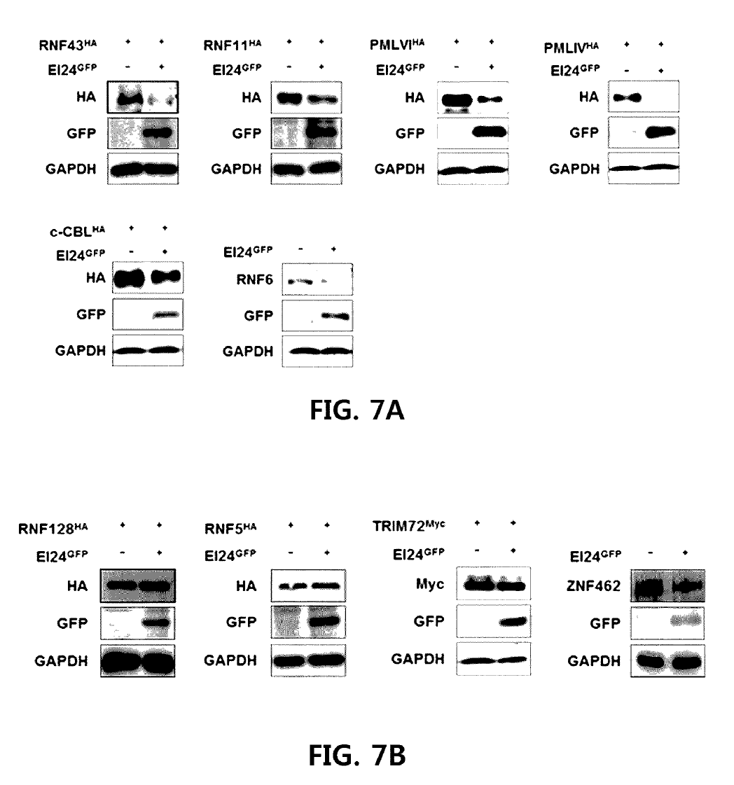

In addition, EI24 was overexpressed in cells randomly selected from among the groups divided by prediction in FIG. 6 and changes in protein level thereof were checked, and, as a result, as illustrated in FIGS. 7A and 7B, it was confirmed that the E3-ligases were degraded by EI24 in Group 1, while the E3-ligases were not degraded by EI24 in Group 2.

In addition, as a result of gene ontology cellular component (GOCC) analysis, as illustrated in FIG. 8, it was confirmed that, in the case of Group 1, EI24 was mainly located in intracellular organelles such as vacuoles, endosomes, lysosomes, cytoskeletons, and ubiquitin-ligase complexes. In contrast, it was confirmed that Group 2 was related to the cytoplasm or the Golgi body around the nucleus.

### EXAMPLE 3

Analysis of Functional Effectiveness of E3-Ligase Degraded by E124

Since the E3-ligases degraded by EI24 was analyzed in terms of molecular informatics, the effectiveness of the E3-ligases (Group 1) degraded by EI24 was analyzed in terms of functionality.

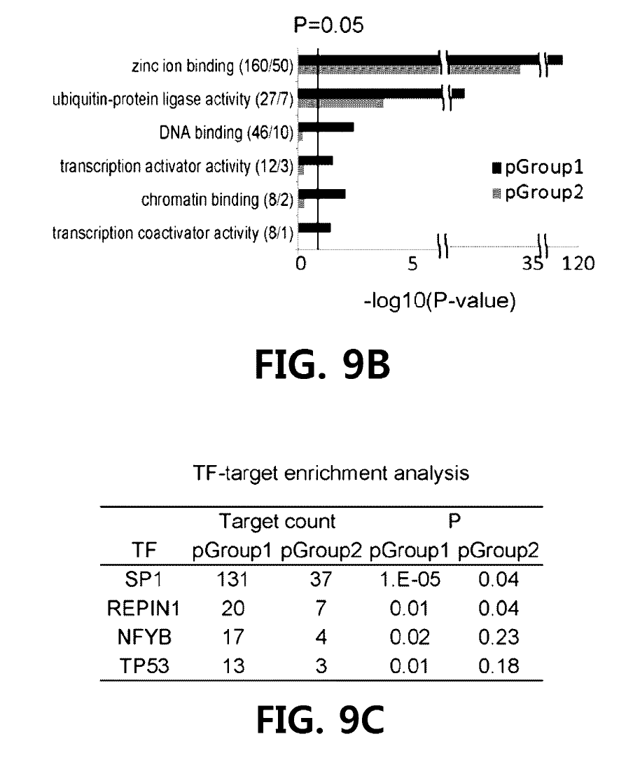

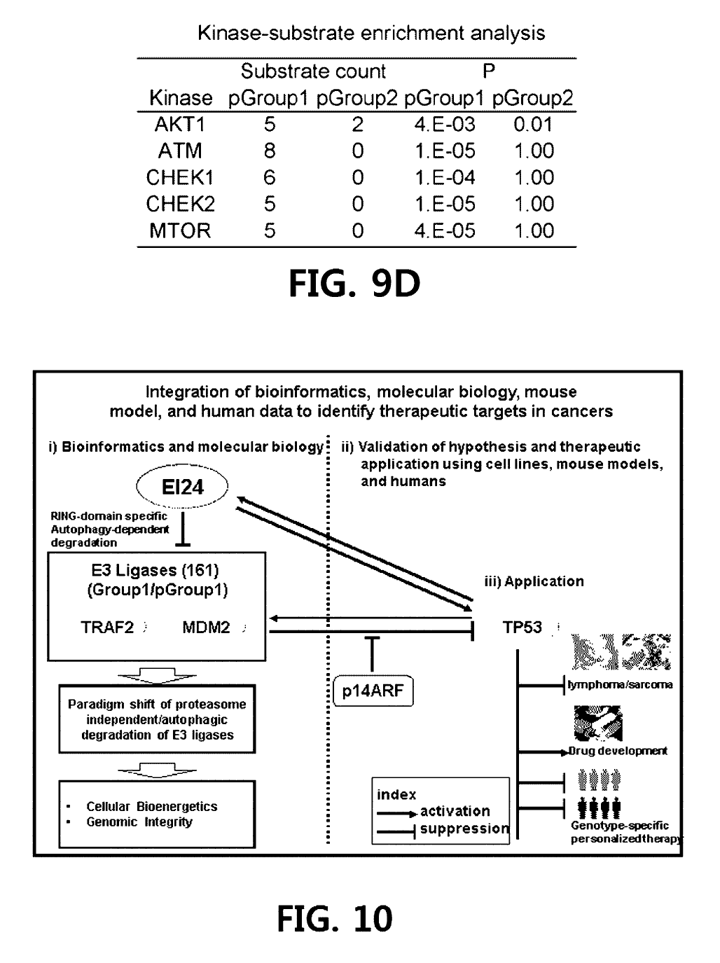

First, as a result of analysis of gene ontology biological functions (GOBPs) and gene ontology molecular functions (GOMFs), as illustrated in FIGS. 9A to 9D, it was confirmed that Group 1 was highly correlated with cell death, cell cycle, changes in histone and chromatin, and RNA metabolism. In addition, it was confirmed that E3-ligases targeted by EI24 were phosphorylated by ATM, CHEKS, mTOR and AKT1 signaling.

When considering the above results all together, as illustrated in FIG. 10, it was confirmed through the experiment that EI24 degraded E3-ligases having the RING domain by inducing an autophagy system, and a new rule therefor through bioinformatics analysis was made. Furthermore, it was shown that EI24 acted as a vital mediator for autophagy and ubiquitin-proteasome systems. It was shown that these mechanisms were closely correlated with various intracellular processes such as bioenergetics, genome conservation, and the like.

### EXAMPLE 4

Analysis of Expression Patterns of ARF and p53 in Cancer

Prior to confirmation of cancer therapeutic effects of EI24 protein of the present disclosure, first, expression patterns of ARF and p53 in cancer were analyzed.

The expression patterns of p53 and ARF genes in 264 patients with sarcomas were analyzed through TCGA database analysis, and, as a result, as illustrated in FIG. 11, it was confirmed that p53 and ARF variations were mutually exclusive. In particular, among a total of the 264 patients with sarcoma, 15% lost ARF functionality, 92% exhibited normal p53 functionality, and 8% exhibited a p53 mutation. From the results, it was confirmed that 92% of sarcoma patients who lost ARF functionality while having normal p53 functionality are curable.

### EXAMPLE 5

Induce Activation of Autophagy According to Presence or Absence of Expression of EI24

Since it was confirmed that the EI24 protein could degrade E3-ligases using the autophagy system, a motif inducing an autophagy mechanism in the EI24 protein was checked. Thus, through sequence alignment analysis of amino acids of EI24, an LIR-motif, which known as a major motif in existing proteins inducing an autophagy mechanism, was confirmed.

As a result, as illustrated in FIGS. 12A to 12C, it was confirmed that the EI24 protein contained an LIR-motif seen in other autophagy mechanism-inducing proteins (see FIGS. 12A to 12C), and, when LC3 and EI24 were overexpressed in 293T embryotic kidney-derived tumor cells, the two proteins interacted with each other. This was also confirmed at an endogenous level. In addition, to check the effectiveness of the LIR-motif of EI24 in the autophagy mechanism, targets were selected. For this, among the amino acid sequences of the EI24 protein, an LIR1 motif with the 81^{st} to 86^{th} amino acid sequence was denoted as #1 LIR-motif, and an LIR2 motif with the 111^{th} to 118^{th} amino acid sequence was denoted as #2 LIR-motif. To check the effectiveness of each of #1 and #2 LIR-motifs, EI24 constructs from which the #1 and #2 LIR-motifs were respectively deleted were prepared and then overexpressed in H1299 lung tumor cells, and it was check whether the autophagy mechanism was activated. Compared to a control, when the #2 LIR motif was deleted, the activity of the autophagy mechanism was decreased, from which it was confirmed that this played an important role in activation of the autophagy mechanism.

In addition, as illustrated in FIG. 13, to check the activation of the autophagy mechanism according to the expression of EI24, the autophagy mechanism was artificially activated by applying Hank's balanced salt solution (HBSS) to 293T embryotic kidney-derived tumor cells, the expression of which was degraded using EI24 siRNA, to be induced into a nutritionally deficient condition, and expression levels of p62 and LC3 were compared with each other. As a result, it was confirmed that, when the expression of EI24 was decreased through the expression levels of p62 and LC3, which are main factors of the autophagy mechanism, the activity of the autophagy mechanism was also decreased.

### Example 6

Confirm Whether MDM2 and p53 Genes Were Expressed According to Activation of Autophagy Mechanism Induced by E124

<6-1> Confirmation of Whether MDM2 was Degraded Through Autophagy by E124

The rule of E3-ligases having the RING domain targeted by EI24 was made in the above example, from which it was confirmed that the 161 E3-ligases were degraded by EI24. Thus, to specify the functional role of EI24 by targeting MDM2, which is one type of E3-ligase, whether MDM2 was expressed according to activation of the autophagy mechanism induced by EI24 was analyzed by comparison.

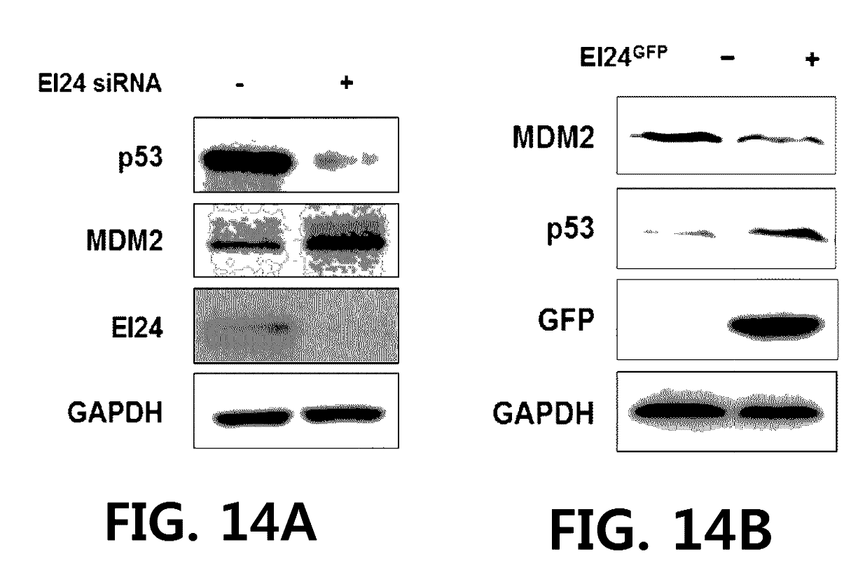

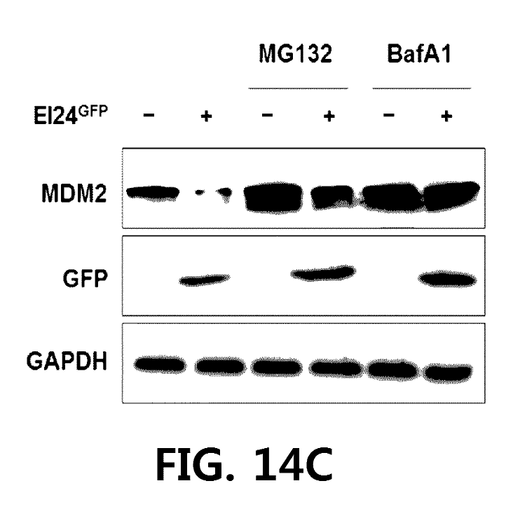

First, as illustrated in FIGS. 14A to 14C, it was confirmed that, when EI24 was overexpressed in HCT116 colorectal tumor cells having the activity of p53, the expression of MDM2 protein was decreased and the expression of p53 protein according thereto was increased. In addition, it was confirmed that these results were induced by the autophagy mechanism.

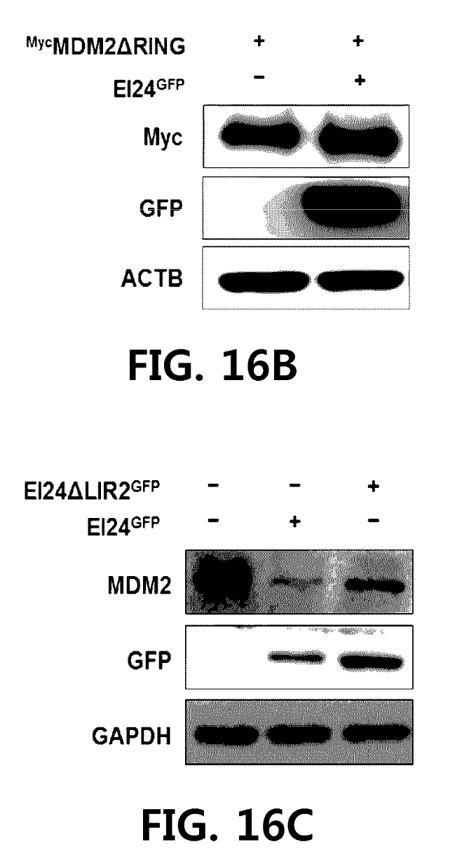

In addition, as illustrated in FIGS. 15A to 15C, it was confirmed that, when MDM2 and EI24 were overexpressed in 293T embryotic kidney-derived tumor cells, the two proteins interacted with each other. This was also confirmed at an endogenous level. In addition, when the RING domain was deleted, MDM2 and EI24 were unable to bind to each other, from which it was confirmed that the RING domain played an important role in interaction between the two proteins. In addition, MDM2 from which the RING domain was deleted and EI24 from which an LIR-motif was deleted were prepared, and, to verify the effectiveness of each of the domain and the motif, it was checked whether MDM2 was expressed according to the expression of EI24. As a result, as illustrated in FIGS. 16A to 16C, when any one of the RING domain of MDM2 and the LIR motif of EI24 was deleted, interaction between the two proteins did not occur, from which it was confirmed that these domain and motif played an important role in interaction between the two proteins.

<6-2> Confirmation of change in degradation level of MDM2 according to change in expression level of E124

EI24 was overexpressed in HCT116 colorectal tumor cells having the activity of p53, and then the cells were treated with cycloheximide, which is a reagent for the inhibition of protein synthesis, for 0 hour, 1 hour, and 3 hours, a change in whether MDM2 was degraded was checked, and expression levels of p53 and MDM2 in HCT116 colorectal tumor cells, the expression of which was degraded using EI24 siRNA, were checked.

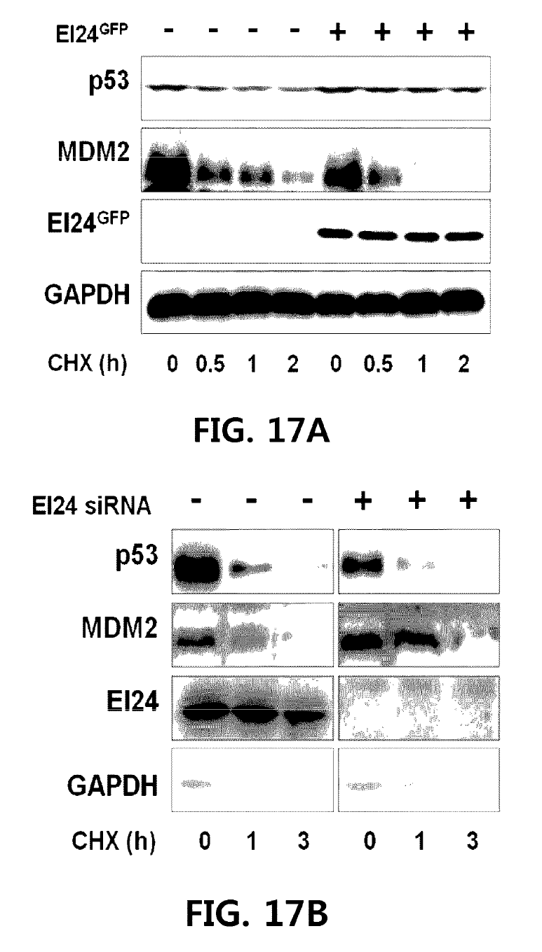

As a result, as illustrated in FIGS. 17A and 17B, it was confirmed that, when treated with cycloheximide, the expression of p53 maintained a certain level without being degraded by MDM2. From the results, it was confirmed that, when the expression of p53 was degraded using EI24 siRNA and then treated with cycloheximide, the expression of p53 was decreased.

In addition, from the results shown in FIGS. 18A and 18B, it was confirmed that, when a ubiquitination degree of p53 of each of p53-activated HCT116 colorectal tumor cells and p53-activated H1299 lung tumor cells was checked, MDM2 was degraded in the EI24-overexpressed cells by an autophagy mechanism, thereby inhibiting ubiquitination of p53 by MDM2.

### EXAMPLE 7

Confirmation of Cancer Cell Proliferation Ability According to the Presence or Absence of E124 in ARF-Expressed or Non-Expressed Cancer Cell Line

<7-1> Confirmation of Expression Patterns of ARF and p53 in Various Types of Cancer Cells

Prior to confirmation of a change in expression level of p53 by EI24, expression patterns and levels of p53 and ARF, which is a protein that regulates the expression of p53, in various types of cancer cells were analyzed.

As illustrated in FIGS. 19A to 19C, as a result of examination of the expression of ARF and p53 using colorectal, liver and lung tumor cells, it was confirmed that both ARF and p53 were expressed in HepG2 liver tumor cells, while ARF was expressed and p53 was not expressed in H1299 lung tumor cells. It was confirmed that HCT116 colorectal tumor cells that had the activity of p53 and did not have the activity of p53 did not have ARF functionality (see FIGS. 19A to 19C).

<7-2> Confirmation of Change in Cell Proliferation Ability According to Overexpression of E124 and/or MDM2 in Cancer Cell Line

Experiments for cell proliferation ability and colony formation were conducted using a group in which EI24 and MDM2 were both overexpressed in ARF-activated HepG2 liver tumor cells and a group in which MDM2 was overexpressed alone therein. In the experiment for measuring the cell proliferation ability, the cell proliferation ability was confirmed by counting the number of cells on day 2, day 4, and day 6, and the colony formation experiment was performed by culturing the cells for 12 hours, staining the cells with crystal violet, and measuring the number of colonies.

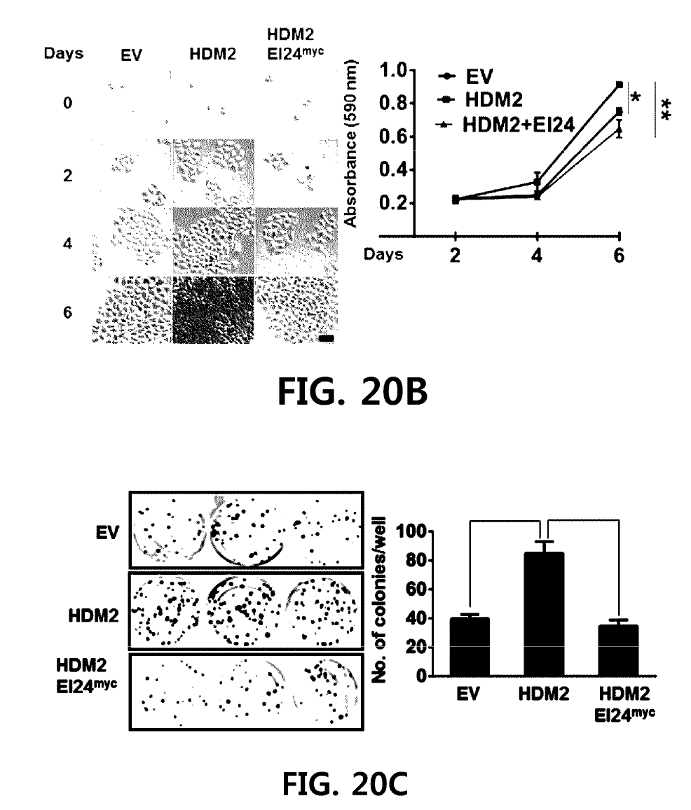

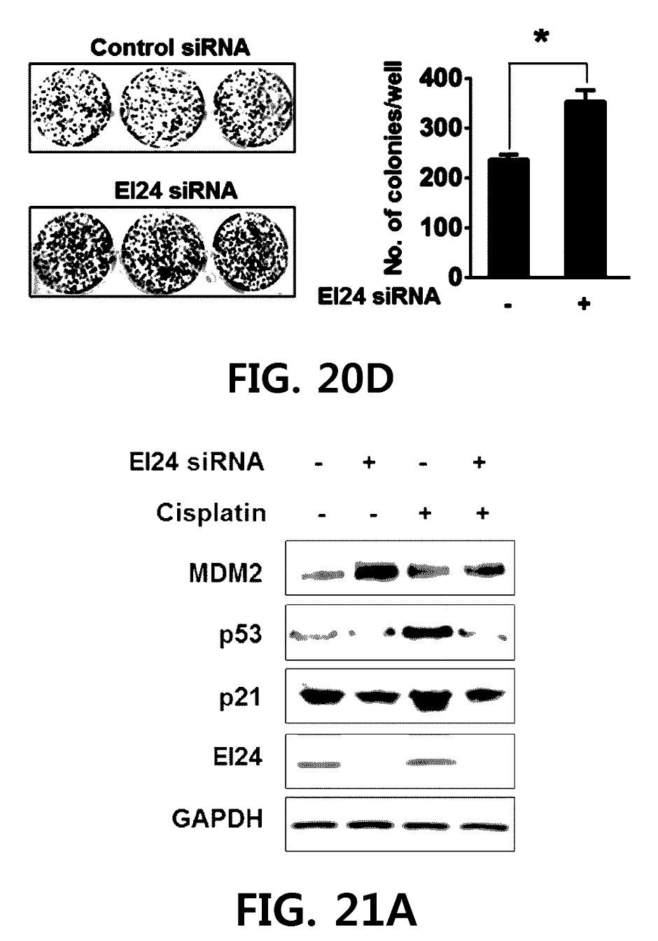

As a result, as illustrated in FIGS. 20A to 20D, it was confirmed that, when MDM2 was overexpressed alone, the cell proliferation ability was high and the colonies were formed in a large number. However, it was confirmed that, when EI24 and MDM2 were both overexpressed, a cell proliferation rate thereof was similar to that of a control and the number of colonies was also decreased.

<7-3> Confirmation of Cell Viability According to E124 Expression Under Genetic Toxic Stress

To comparatively analyze the presence or absence of expression of EI24-MDM2-p53 signaling under genetic toxic stress, the expression of EI24 in HCT116 colorectal tumor cells having the activity of p53 was decreased using EI24 siRNA and the cells were treated with cisplatin for 24 hours to induce stress conditions.

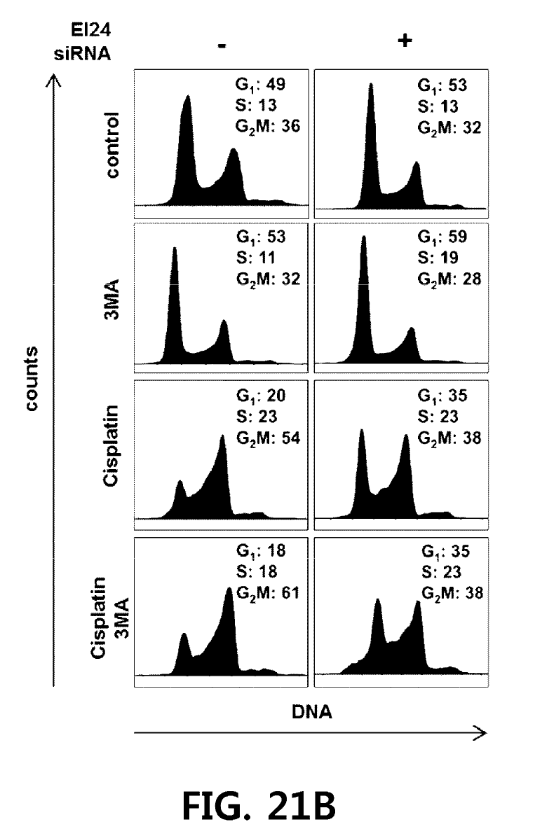

As a result, as illustrated in FIGS. 21A and 21B, it was confirmed that, when the expression of EI24 was decreased under genetic toxic stress, p53 was not maintained at a certain level and, accordingly, this even affected the expression of p21, which is a transcription factor of p53. From the results, it was confirmed that, when p53 was resistive to genetic toxicity, a mechanism for degradation of MDM2 by EI24 was important.

In addition, to examine the correlation between the mechanism confirmed in the above-described example and ARF signaling, the physiological role of an EI24-MDM2-p53 complex was comparatively analyzed in the absence of ARF activity, and EI24 was overexpressed in HCT116 colorectal tumor cells with or without p53 activity, and colony formation abilities thereof were examined. In addition, the cultured cells were stained with crystal violet after 12 days and the number of colonies was measured.

As a result, as illustrated in FIGS. 22A to 22C, it was confirmed that the number of colonies according to the expression of EI24 was decreased in the HCT116 colorectal tumor cells with p53 activity. In contrast, it was shown that, even though EI24 was overexpressed in the HCT116 colorectal tumor cells without the activity of p53, there was no difference in the number of colonies, as compared to a control. From the results, it was confirmed that, when EI24 acted as a tumor inhibitor, such as a role in degrading MDM2, or the like, the expression of p53 should normally occur.

### EXAMPLE 8

Analysis of Tumor Formation Inhibitory Ability According to Expression of E124 in Mouse Model

To confirm whether the same phenomenon was observed in vivo based on the above in vitro experimental results, HCT116 colorectal tumor cells with or without the activity of p53, or MDA-MB-231 cells, which are a triple negative breast cancer cell line, were transformed with an EI24 overexpression vector and xenografted into immunodeficient mice, and then the presence or absence of tumor formation was observed for 20 days. Cancer tissue formed in each mouse was extracted and the expression of EI24, ARF, MDM2, and p53 according to tissue was examined.

As a result, as illustrated in FIGS. 23A to 23D, it was confirmed that the same results as those in vitro confirmed in the above examples were obtained, the formation of tumors according to the expression of EI24 was decreased in the presence of the activity of p53. In contrast, it was confirmed that, in the absence of the activity of p53, EI24 could not properly function as a tumor inhibitor. In addition, it was confirmed that, when the presence or absence of the activity of each gene was examined in cancer tissue, the activity of MDM2 was decreased in both cancer caused by the EI24-overexpressed cells with the activity of p53 and cancer caused by the EI24-overexpressed cells without the activity of p53, and the activity of p53 was increased by the expression of EI24 only in cancer caused by the cells with the activity of p53.

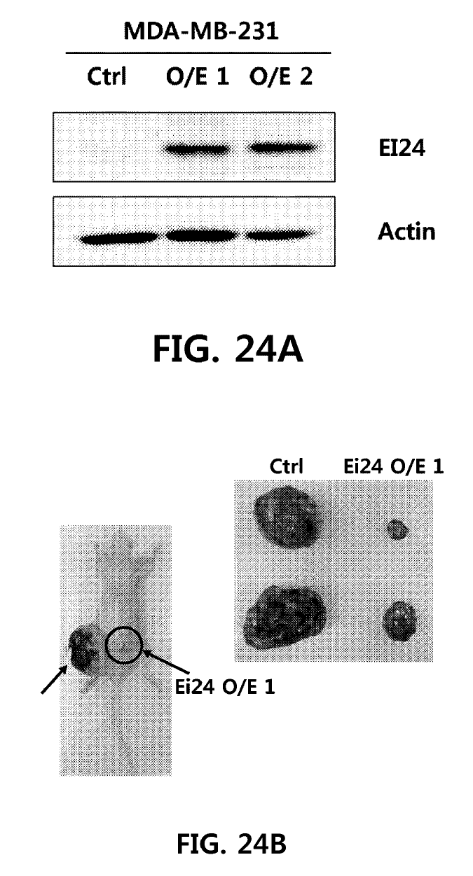

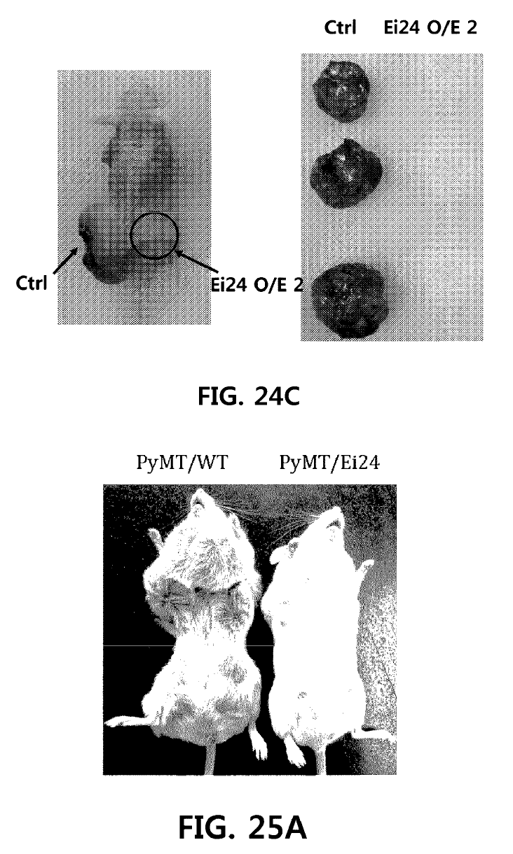

In addition, as illustrated in FIG. 24A-24C, it was confirmed that a tumor formation level was significantly decreased in the EI24-overexpressed cell line compared to a control even in mice xenografted with MDA-MB-231 cells, which are a triple negative breast cancer cell line, from which it was confirmed that EI24 may have a cancer inhibitory effect on triple negative breast cancer.

Similarly, overexpression of EI24 was induced in PyMT mice, which are a breast cancer mouse model, and then the formation and size of breast cancer thereof were compared with those of mice on which the overexpression of EI24 was not induced.

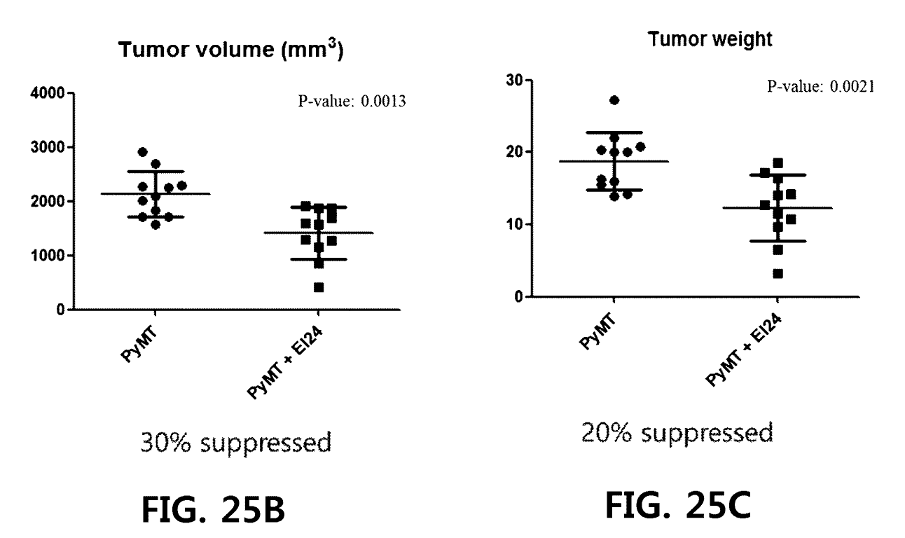

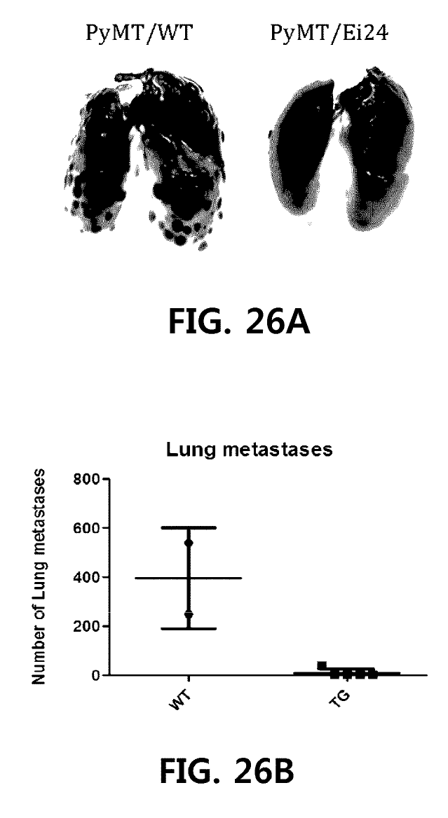

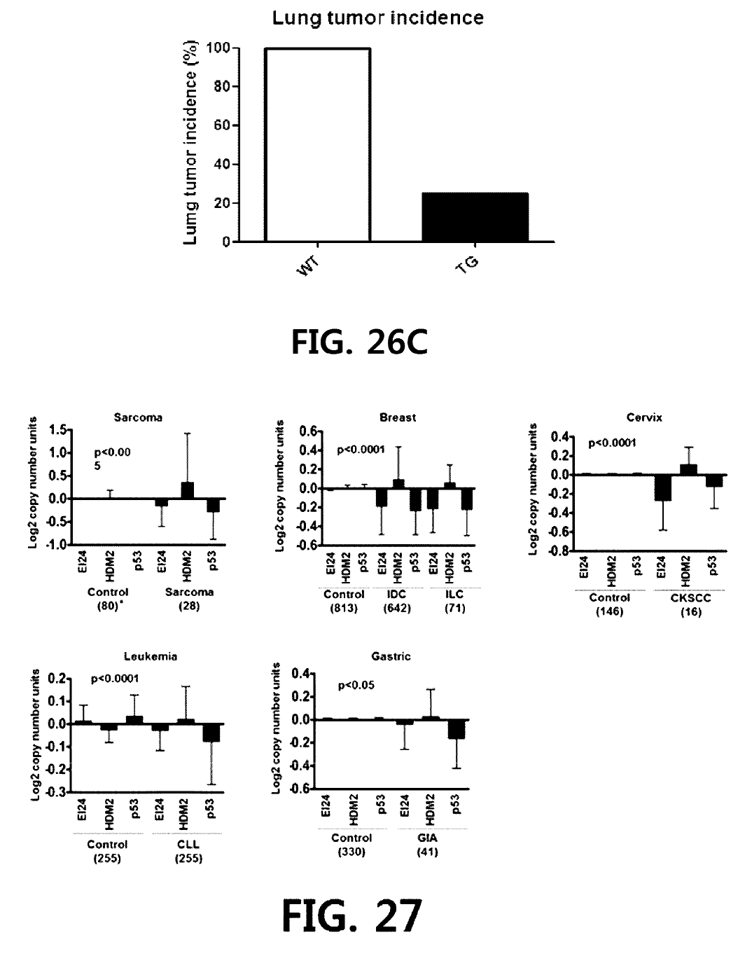

As a result, as illustrated in FIG. 25A-25C, it was confirmed that the formation level and size of breast cancer were decreased in EI24-overexpressed mice. In addition, as a result of examining whether there was metastasis to lung, as illustrated in FIG. 26A-26C, it was confirmed that metastasis of tumors to the lungs was observed in the mice on which the overexpression of EI24 was not induced, while metastasis to lung exhibited no significant level in the EI24-overexpressed mice.

### EXAMPLE 9

Whether E124, MDM2, and p53 Were Expressed in Various Types of Cancer

To more particularly examine the correlation among EI24, MDM2, and p53 confirmed in the present disclosure, the expression of EI24, MDM2, and p53 was examined in tissue samples of patients with breast cancer, sarcomas, leukemia, and gastric cancer.

As a result, as illustrated in FIG. 27, it was confirmed that EI24 DNA copy number positively interacted with p53 and negatively interacted with HDM2.

### EXAMPLE 10

Confirmation of E124 Lower Protein Involved in Cancer Inhibitory Effects by E124 Protein

<10-1> Confirmation of Lower Protein, Expression Level of Which was Varied by E124

Since it was confirmed through Example 8 above that, when EI24 was overexpressed in PyMT mice, the growth of breast cancer was inhibited, lower genes or proteins involved in EI24-mediated cancer inhibition were investigated.

First, breast cancer tissue samples were extracted from a breast cancer mouse model PyMT on which the overexpression of EI24 was induced and mice on which the overexpression of EI24 was not induced, and then RNA was isolated from each breast cancer tissue sample and changes in gene expression level were examined by microarray assay.

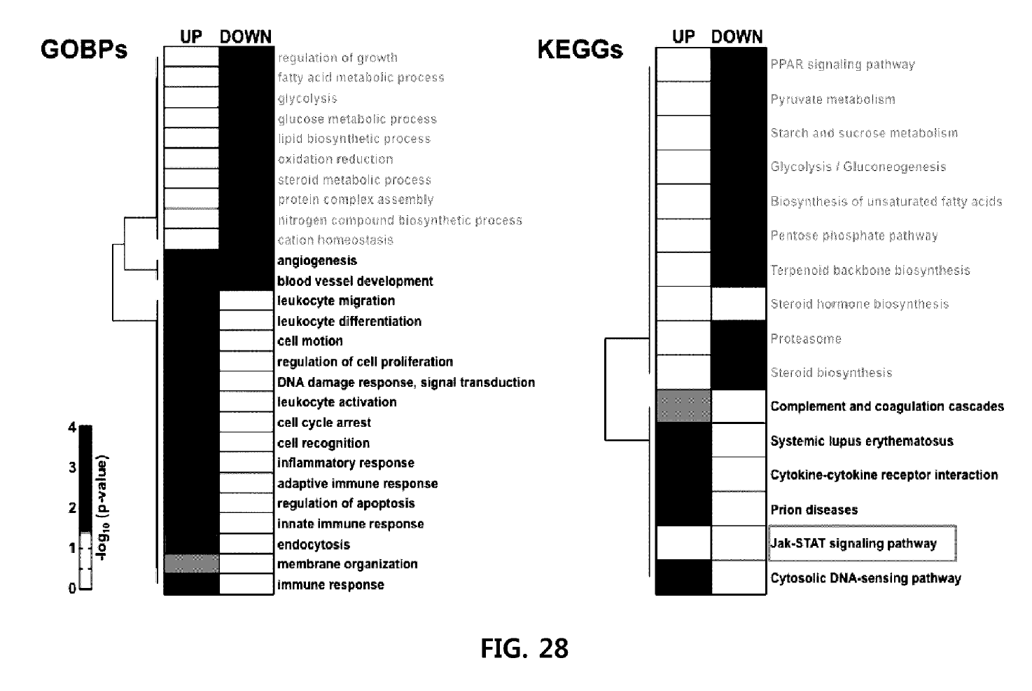

As a result, as illustrated in FIG. 28, it was confirmed that the expression of genes belonging to metabolism, adipogenesis, calcium synthesis, immune responses, and JAK-STAT signaling pathway was regulated.

Among these, to reconfirm proteins, expression levels of which were varied by EI24, PyMT gene and/or EI24 was overexpressed in an MCF7 cell line, and then expression levels of intracellularly expressed proteins were examined by western blotting.

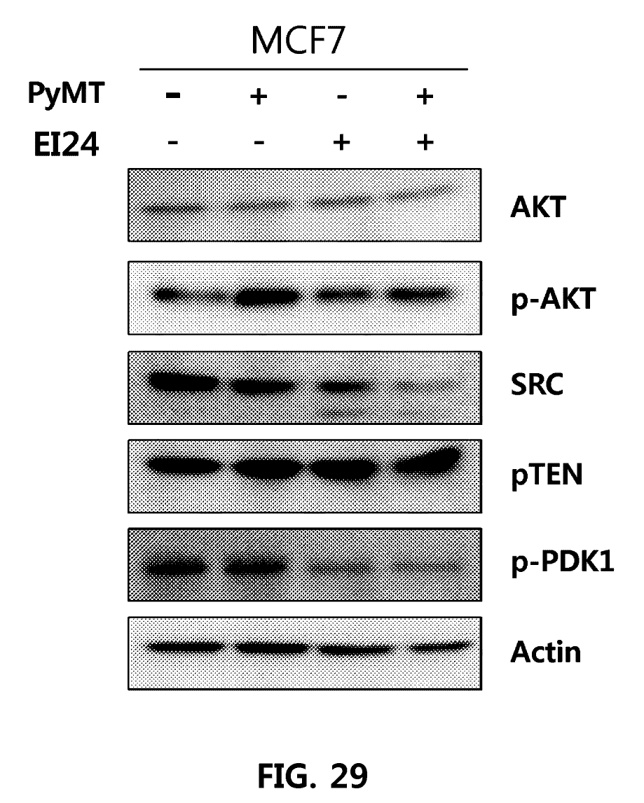

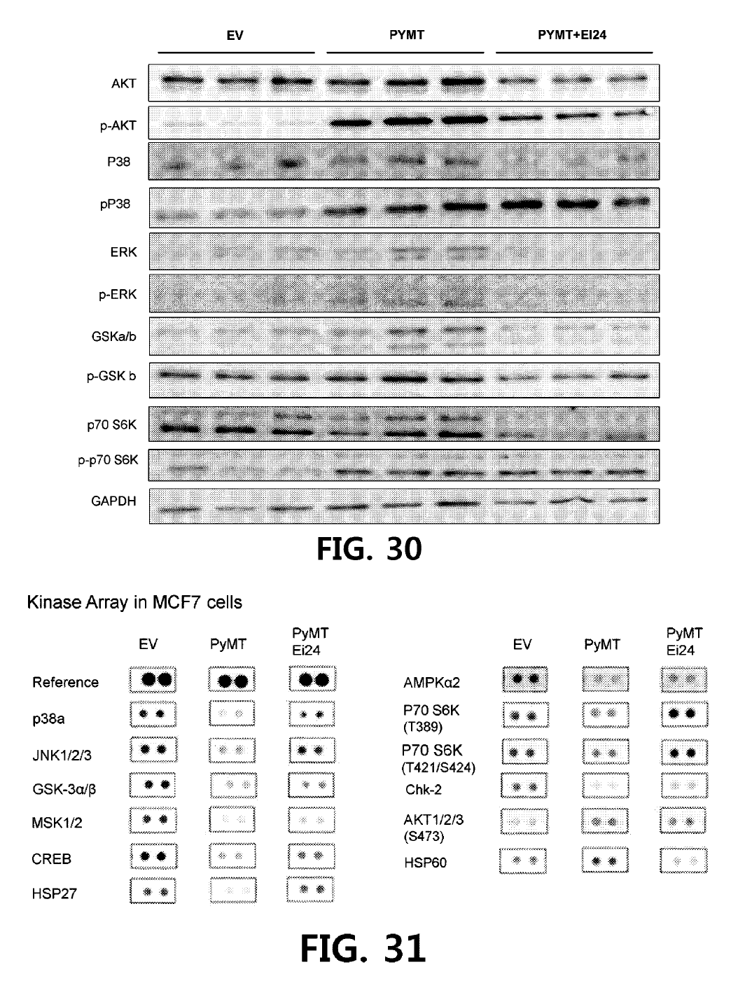

As a result, as illustrated in FIGS. 29 and 30, it was confirmed that expression levels of AKT, p-AKT, SRC, pTEM, and p-PDK1, which are proteins belonging to the AKT pathway, P38, pP38, ERK, p-ERK, GSKa/b, p-GSK b, p70 S6K, and p-p70 S6K, were significantly decreased.

From the results, it was confirmed that EI24 targeted AKT and p-AKT and thus, when EI24 was overexpressed, the expression levels of AKT and p-AKT were decreased, from which it was confirmed that EI24 acted as a tumor inhibitor through the AKT pathway.

<10-2> Confirmation of Lower Proteins, Activity Levels of Which Were Varied by E124

In addition to the EI24 lower proteins, expression levels of which were decreased by the overexpression of EI24, proteins, activity levels of which were varied, were examined. First, EI24 and PyMT were overexpressed in breast cancer cells, and thus proteins exhibiting changes in activity level were screened through kinase array.

As a result, as illustrated in FIG. 31, it was confirmed that, when protein synthesis in a cancer cell line on which PyMT was overexpressed alone was compared with protein synthesis in a cancer cell line on which PyMT and EI24 were both overexpressed, the activity levels of proteins, i.e., p38a, JNK1/2/3, GSK-3α/β, MSK1/2, CREB, HSP27, AMPKa2, P70 S6K(T389), P70 S6K(T421/S424), and Chk-2, were increased according to the expression of EI24. In contrast, it was confirmed that the activity level of HSP60 was decreased according to the expression of EI24.

EI24 according to the present disclosure can degrade MDM2 through an autophagy system and, accordingly, can recover an MDM2-induced decrease in p53 activity. This can occur regardless of ARF signaling, and thus enables the recovery of p53 functionality through the expression of the EI24 protein even under ARF activity-inhibited or suppressed cancer environments, and can provide cancer treatment effects. Thus, activation of an EI24-mediated mechanism of the present disclosure can play an important role in development of a variety of cancer therapeutic agents.

It will be apparent to those skilled in the art that various modifications can be made to the above-described exemplary embodiments of the present disclosure without departing from the spirit or scope of the invention. Thus, it is intended that the present disclosure covers all such modifications provided they come within the scope of the appended claims and their equivalents.

What is claimed is:

1. A method of treating cancer, comprising: administering a pharmaceutically effective amount of EI24 protein or a fragment thereof, or an expression vector comprising a nucleotide sequence encoding the EI24 protein or the fragment thereof; or an activating agent increasing expression or activity of EI24, to an individual in need thereof.

2. The method of claim 1, wherein the EI24 protein is a protein having an amino acid sequence of SEQ ID NO: 1.

3. The method of claim 1, wherein the nucleotide sequence comprises a polynucleotide having a base sequence of SEQ ID NO: 2.

4. The method of claim 1, wherein, in the cancer, ARF gene is non-activated.

5. The method of claim 1, wherein, in the cancer, expression and activity levels of p53 protein or p53 gene are similar to those of normal cells.

6. The method of claim 1, wherein the cancer comprises any one or more selected from the group consisting of breast cancer, cervical cancer, leukemia, gastric cancer, sarcomas, liver cancer, lung cancer, colorectal cancer, and renal carcinoma.

7. A method of screening a candidate for cancer treatment, the method comprising:
treating a patient-derived sample comprising cells that under-express or are incapable of expressing EI24 protein, or a sample comprising EI24 protein, with test substances;
measuring an expression level or activity level of the EI24 protein in the treated sample; and
selecting a test substance that enables the measured expression level or activity level of the EI24 protein to be increased compared to a control not treated therewith.

8. The method of claim 7, wherein the measuring of the expression level is performed using any one or more selected from the group consisting of immunoprecipitation, radioimmunoassay (MA), enzyme linked immunosorbent assay (ELISA), immunohistochemistry, western blotting, and fluorescence activated cell sorting (FACS).

9. The method of claim 7, wherein the measuring of the activity level is performed using any one or more selected from the group consisting of sodium dodecyl sulfate (SDS)-polyacrylamide gel electrophoresis (PAGE), immunofluorescence, ELISA, mass analysis, and a protein chip.

10. The method of claim 7, wherein, in the cancer, ARF gene is non-activated.

11. The method of claim 7, wherein, in the cancer, expression and activity levels of p53 protein or p53 gene are similar to those of normal cells.

12. The method of claim 7, wherein the cancer comprises any one or more selected from the group consisting of breast cancer, cervical cancer, leukemia, gastric cancer, sarcoma, liver cancer, lung cancer, colorectal cancer, and renal carcinoma.

13. A method of screening a candidate for cancer treatment, the method comprising:
treating a patient-derived sample comprising cells that under-express or are incapable of expressing E124 protein, or a sample comprising E124 protein, with test substances;
measuring an expression level or activity level of a lower protein of the E124 protein in the treated sample; and
selecting a test substance by comparing the measured expression level or activity level of the lower protein of the E124 protein with that of a control not treated therewith.

14. The method of claim 13, wherein the lower protein of the E124 protein comprises at least one selected from a) below and b) below:
a) any one or more proteins selected from the group consisting of AKT, p-AKT, SRC, pTEM, p-PDK1, P38, pP38, ERK, p-ERK, GSKa/b, p-GSK b, p70 S6K, p-p70 S6K, and HSP60, the proteins with a decreasing expression or activity level; and
b) any one or more proteins selected from the group consisting of p38a, JNK1/2/3, GSK-3α/β, MSK1/2, CREB, HSP27, AMPKa2, P70 S6K(T389), P70 S6K(T421/S424), and Chk-2, the proteins with an increasing expression or activity level.
